(19) United States
(12) Patent Application Publication
(10) Pub. No.: US 2015/0140578 A1
(43) Pub. Date: May 21, 2015

(54) METHODS AND DEVICES FOR SAMPLE TESTING AND EVALUATION

(71) Applicant: Arryx, Inc., Braintree, MA (US)

(72) Inventor: Daniel M. Mueth, Chicago, IL (US)

(21) Appl. No.: 14/404,210

(22) PCT Filed: May 28, 2013
(86) PCT No.: PCT/US13/42885

§ 371 Date: Nov. 26, 2014

Related U.S. Application Data
(60) Provisional application No. 61/652,815, filed on May 29, 2012.
(60) Provisional application No. 61/692,113, filed on Aug. 22, 2012.

(51) Int. Cl. G01N 33/80

(52) U.S. Cl. CPC G01N 33/80; G01N 2800/22
USPC 435/7.25

(57) ABSTRACT

Methods and devices for evaluating a sample from a subject for the presence of a mixed population of cells, e.g., a sample having more than one distinct populations of cells, e.g., red blood cells (RBCs). Also included are methods and devices for Coombs crossmatch and DAT testing.

### RELATED APPLICATIONS

This application claims the benefit of U.S. Provisional Application No. 61/652,815, filed May 29, 2012, and U.S. Provisional Application No. 61/692,113, filed Aug. 22, 2012, the contents of all of which are incorporated herein by reference in their entirety.

### BACKGROUND

Blood type testing is a set of tests which include testing for major red blood cell blood group antigens (A, B, D) (referred to as forward blood typing) and minor red blood cell blood group antigens (e.g., c, C, e, E, K, Jk^{a}, Jk^{b}, Fy^{a}, Fy^{b}, Pl, M, N, S, and s), along with other tests. These antigen tests generally involve the measurement of hemagglutination of the red cells being tested after incubation with antibodies specific to the blood group antigen of interest, and possibly additional steps and reagents, such as incubation with anti-human globulin (AHG). Generally, all red blood cells of an individual have identical antigen expression, so one would expect all of the cells to tend to agglutinate or none of them to. Thus, a test result may be binary, indicating if the individual is positive or negative for the antigen. A further refinement of the test is to grade the reaction strength, however it is still generally expected that for most individuals the red cells all express, or do not express, a given antigen.

There are, however, cases wherein a patient's blood sample may have two populations of cells with differing antigen expression. For example, a patient who has recently been transfused with red blood cells may have two populations of red cells with different antigen expression (e.g. an A+ patient transfused with O− blood). In traditional manual tube testing, testing for the A antigen using the anti-A antibody would lead to one population agglutinating while the other population does not agglutinate. The correct test result would be reported as “mixed field”, indicating that some cells are agglutinating and some are not.

The need to improve quality, automation, consistency, and throughput along with the desire to minimize the possibility of human errors has driven hospital blood banks to automate their type testing. Two dominant technologies are employed widely. The first, gel technology, is able to correctly report “mixed field” for such samples, but is slow and relies heavily on a large number of expensive liquid reagents. The second set of technologies are solid phase technologies which differ somewhat from each other, but generally do not correctly report mixed field samples as “mixed field”.

The need exists for developing tests that reliably and efficiently evaluate samples containing a mixed population of cells, e.g., mixed population of red blood cells.

### SUMMARY

The invention provides, inter alia, methods, devices, kits, and assays for evaluating a sample (e.g., a plasma sample, a serum sample, or a whole blood sample) from a subject for the presence of a mixed population of cells, e.g., a sample having more than one distinct populations of cells, e.g., red blood cells (RBCs). The invention also provides, inter alia, methods, devices, kits, and assays for performing direct agglutination tests and indirect agglutination tests wherein the capture agent is disposed on a solid substrate, e.g., on the surface of a test chamber, e.g., a well or tube. Thus, methods, devices, kits, and assays that include one or more of the aforesaid embodiments are disclosed. The present invention can be applied to blood typing, including, but not limited to, forward typing, minor antigen typing, extended phenotyping and indirect agglutination tests (IAT) and direct agglutination tests (DAT).

Accordingly, the invention features, a method of preparing and/or evaluating a sample for the presence of one or both of a first population of cells (e.g., RBCs) and a second population of cells (e.g., RBCs). The method allows the evaluation of a sample for the presence of a mixed population, i.e., a sample having both a first population and a second population of cells, wherein the first and second population differ, e.g., by a surface characteristic or feature, e.g., a surface antigen. In an embodiment, cells of the first population present a first cell surface antigen, e.g., a RBC antigen, and cells of the second population of cells are devoid or substantially devoid of the cell surface antigen.

The method comprises:

(a) providing a capture agent disposed on a substrate,

wherein the capture agent binds the surface feature, e.g., an antigen on cells of the first population but not cells of the second population, and

wherein the substrate is configured such that cells that do not bind to the capture agent migrate to a negative readout region (NRR);

(b) contacting the sample with the capture agent under conditions sufficient for binding, e.g., the formation of an immune complex, between the capture agent and a cell of the first population of cells, which binding occur, e.g., in a positive readout region (PRR);

(c) providing conditions that allow cells of the of the second population that do not bind to the capture agent to migrate into a NRR;

(d) classifying (e.g., scoring) the PRR for presence of cells (In an embodiment, classifying can be done by determining if the number of cells in the PRR meets a predetermined reference value(s). In an embodiment, classifying can be done by gauging the number of cells in the PRR. In an embodiment, meeting a first value (e.g., a range) indicates a “positive.” In an embodiment, meeting a second value (e.g., range) indicates “non-positive.” In embodiments, the first, the second, or both values (e.g., ranges) are used. The value(s) (e.g., range(s)) can, e.g., be determined by running calibration standards having known amounts of cells from the first and known amounts of cells from the second population. In another embodiment, the value(s) (e.g., range(s)) can be determined by running samples from subjects having only one of the two populations or known to have a mixed population of cells);

(e) classifying (e.g., scoring) the NRR as positive or non-positive for the presence of cells;

wherein,
(i) PRR+ and NRR− is indicative of the sample lacking the second population; In an embodiment, a PRR+ includes a positive classification or high score for the cell presence in the PRR, and a NRR− includes a negative classification or low score for the cell presence in the NRR;
(ii) PRR− and NRR+ is indicative of a sample lacking the first population. In an embodiment, a PRR− includes a negative classification or low score for the cell presence in the PRR, and a NRR+ includes a positive classification or high score for the cell presence in the NRR;
(iii) PRR+ and NRR+ is indicative of a sample having both the first population and the second population. In an embodiment, a PRR+ includes a positive classification or high score for the cell presence in both the PRR and the NRR,
   thereby preparing and/or evaluating a sample for a mixed population of RBCs.

In another aspect, the invention features a method of preparing and/or evaluating a sample for a mixed population of RBCs, e.g., a sample comprising a first population of RBCs and a second population of RBCs, wherein cells of said first population comprise a surface feature, e.g., a surface antigen, and cells of said second population are substantially devoid of said surface feature. The method comprises:

(a) providing a capture agent disposed on a substrate,

wherein the capture agent binds the surface feature, e.g., an antigen on cells of the first population but not cells of the second population, and

wherein the substrate is configured such that cells that do not bind to the capture agent migrate to a negative readout region (NRR);

(b) contacting the sample with the capture agent under conditions sufficient for binding, e.g., the formation of an immune complex, between the capture agent and a cell of the first population of cells, which binding occur, e.g., in a positive readout region (PRR);

(c) providing conditions that allow cells of the of the second population that do not bind to the capture agent to migrate into a NRR;

(d) performing one or both of
(i) classifying the PRR as:
   single or high positive (which is indicative of a sample lacking the second population);
   mixed or low positive (which is indicative of a sample having both the first and second populations); or
   negative, (which is indicative of a sample lacking the first population); or
(ii) classifying the NRR as:
   single or high positive (which is indicative of a sample lacking first population);
   mixed or low positive (which is indicative of a sample having both the first and second populations); or
   negative (which is indicative of a sample lacking the second population)
      thereby preparing and/or evaluating a sample, e.g., for a mixed population of RBCs.

In an embodiment, the methods can be done by determining if the number of cells in the PRR (or NRR) meets a predetermined reference value(s) for each of the three possible outcomes, single or high positive; mixed or low positive; and negative. The value can incorporate an appreciation of the total number of cells in the assay, which allows for determination based on only the PRR or NRR. For example, one would see a positive value for PRR both in a sample having no second population and in a sample having both populations, but the positive value in the former to be a higher proportion of cells in the sample than in the case of the latter.

In an embodiment, the method includes evaluating the sample for the presence of a population having a second surface feature and a population devoid of or substantially devoid of the second surface feature.

In an embodiment, the method includes determining if the first and second cell surface features are on the same or different populations.

In an embodiment, the cells in both the first and second populations are RBCs. The surface feature present on the cells of the first population and not present on, or substantially devoid from, the cells of the second population is a surface antigen from Table 1. In an embodiment the capture agent, e.g., an antibody, is specific for the surface antigen. Thus, the method can detect the presence or absence of populations having a different surface antigen, and can detect the presence of a mixed population.

In an embodiment, the surface feature present on the cells of the first population and not present on, or substantially devoid from, the cells of the second population is a RBC blood group antigen, e.g., A, B or D. In an embodiment, the capture agent, e.g., an antibody, is specific for the RBC blood antigen, e.g., for an A, B or D antigen. Thus, the method can detect the presence or absence of populations having a different RBC blood group antigen, and can detect the presence of a mixed population.

In an embodiment, the surface feature present on the cells of the first population and not present on, or substantially devoid from, cells of the second population is a minor RBC blood group antigen. E.g., the antigen is: a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. Thus, the method can detect the presence or absence of populations having a different minor RBC blood group antigen, and can detect the presence of a mixed population.

In an embodiment, the sample is from a subject that has had a blood transfusion, e.g., a recent blood transfusion, e.g., a transfusion within 1, 5, 10, 20, 30 60, or 90 days of taking of the sample.

In an embodiment, the sample is from a subject that has had a bone marrow, or other tissue, transplant.

In an embodiment, the sample is from a subject that is a hematopoietic chimera, e.g., a subject having naturally occurring chimerism (e.g., arising from the fusion of more than one embryo into a single embryo or the exchange of cells between embryos) or having chimerism arising from a transplant.

The method can also be used to evaluate a sample for the presence of a population of cells having IgG antibodies bound to the surface of the cells. In such embodiments the surface feature to which the anti-surface antigen-antibody is bound is a surface antigen that is present on cells of a first population. Cells of the second population are devoid, or substantially devoid of the anti-surface antigen-antibody. While not wishing to be bound by theory, it is believed the lack of antibodies is due to the lack of the surface antigen on the cells of the second population.

Thus, in an embodiment, the capture agent binds an anti-surface antigen antibody bound to cells of the first population.

In an embodiment the capture agent comprises an antibody, e.g., an anti-human globulin (AHG). In an embodiment the capture agent comprises one or both of anti-C3D and anti-IgG antibodies.

In an embodiment, the sample: (1) is from a subject that has had a HTR (hemolytic transfusion reaction), (2) is from a subject that has had a DHTR (delayed hemolytic transfusion reaction), (3) is from a newborn having its RBCs coated with IgG-class antibodies from the mother, such as anti-K, anti-E, or other IgG-class antibodies to major or minor antigens which are present on the newborn's RBCs but which the mother has formed IgG-class antibodies to, (4) is from a subject that has autoantibodies which attach to it's own cells, (5) is from a subject other than one with HTR, where shortened cell life occurs due to antibodies from the subject coating transfused cells, (6) is from a subject that has drug-induced non-specific binding of antibodies to a its's RBCs, (7) is from a subject that has other non-specific binding of antibodies to a it's RBCs, or (8) is from a subject that has other causes of anti-RBC antibodies.

In an embodiment, the surface feature present on the cells of the first population and not present on, or substantially devoid from, the cells of the second population is an antibody that binds A, B or D antigen.

In an embodiment, the surface feature present on the cells of the first population and not present on, or substantially devoid from, cells of the second population is an antibody to a minor RBC blood group antigen. E.g., the antigen is: a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment, the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. Thus, the method can detect the presence or absence of populations having a different minor RBC blood group antigen.

In an embodiment, the capture agent comprises molecules that bind to a surface feature, e.g., RBC antigen, e.g., a protein, a peptide or a carbohydrate. In one embodiment, capture agent is an anti-RBC antigen antibody (e.g., an IgG or an IgM, or a combination thereof). In other embodiments, the capture agent comprises a plant-derived binding agent, e.g., a lectin. In other embodiments the capture agent comprises an anti-IgG antibody.

In an embodiment, the capture agent, e.g., antigen antibody, is disposed on the inner surface of a chamber, e.g., well or tube.

In an embodiment, the amount of sample cells contacted with the substrate is such that, if layered onto the substrate, e.g., the portion of the substrate coated with capture agent, it would form less than a monolayer of sample cells.

In an embodiment, the amount of sample cells contacted with the substrate is such that the binding sites of the capture agent are not saturated, e.g., less than 90, 80, 70, 60 or 50% of the available sites on the capture agent are bound to sample cells.

In an embodiment, the amount of sample cells contacted with the substrate is such that the sample cells of the first population of cells do not migrate into the negative readout region.

In an embodiment, positive and negative readout regions are imaged to provide a result. In an embodiment, the image analysis can identify (a) whether there is a pellet or other concentration of cells in the negative readout region, e.g., near the center or bottom of a chamber, and (b) whether there is a preselected number cells away from negative readout region.

In an embodiment, the cells in the negative readout region can be imaged by illuminating from above with visible light and imaging from below with a camera under conditions that allow resolution of a darker region of appropriate size, e.g., several millimeters in diameter. In an embodiment cells away from the negative readout position can be imaged, e.g., with a high-resolution imaging system which can resolve individual cells and determining the number density of cells at a certain distance from the negative readout region, such as in the annulus ranging from 0.5r to 0.75r from the negative readout region center, where r is the chamber radius.

In an embodiment, a threshold cell concentration is established by testing “mixed field” samples.

In an embodiment, the substrate is configured such that the applied acceleration allows cells of the first population to be distinguished from cells of the second population. E.g., applied acceleration results in migration of uncomplexed cells (cell is the second population) into a negative readout region. The negative readout position can, e.g., be at the bottom of a chamber (e.g., a well or a tube), or distal (relative to the direction of the applied acceleration) to a portion of the substrate to which capture agent is disposed. In an embodiment, the detection of the presence of uncomplexed cells (e.g., a negative readout) is correlated with the absence of binding between the cells and the capture agent. In certain embodiments, the negative readout is characterized by a button or a pellet of cells. Exemplary schematics of negative readouts are shown in FIGS. 1D and 2D as samples E and F.

In an embodiment, the substrate includes inner surface of a round-bottomed chamber and the negative readout region is, e.g., at the bottom of the round-bottom chamber.

In an embodiment, the substrate includes inner surface of a conical-bottomed chamber and the negative readout region is, e.g., at the bottom of the conical-bottom chamber.

In an embodiment, substrate is substantially planar substrate.

In an embodiment, the substrate is substantially planar and the angle between the substrate and the direction of applied force, e.g., centrifugal, gravitational, fluid magnetic, electric or fluid, force, that causes migration of detection agent, is non-orthogonal or other than 90 degrees (in the case of a centrifugally applied force, theta, the angle formed by the substrate and a line perpendicular to the direction of centrifugal force, is nonzero).

In one embodiment, the negative readout region is located on the substrate. In other embodiments, the negative readout region is not located on the substrate.

In other embodiments, the method includes comparing a value for the amount of cells present in the negative readout region with a pre-selected criterion.

In other embodiments, the method includes comparing a value for the amount of cells present in the positive readout region with a pre-selected criterion.

In an embodiment, the negative readout region is disposed in a chamber, e.g., a well or tube.

In an embodiment, the chamber is disposed on a carrier, e.g., a multi-chamber or multi-well plate, e.g., a 96 well plate.

In an embodiment, the angle between the carrier and the direction of force is non normal, e.g., between 25-5, 20-7.5, or 10 degrees.

In an embodiment, differential migration of RBCs not complexed with the capture agent (cells of the second population) relative to the complexed cells (cells of the first population) across the substrate, results in migration of cells of the second population migrating into the negative readout region.

### Applied Force and Other Embodiments

In an embodiment, migration of cell of the second population is effected by applying acceleration, e.g., centrifugal, fluid magnetic, electric or fluid, that causes migration of uncomplexed cells.

In certain embodiments, two different forces are applied, a first force to provide force normal to the substrate or a surface and a second force to provide force tangential to the substrate or a surface. In one embodiment, the first force, e.g., a magnetic force, is applied to produce force normal to the substrate or a surface on a detection agent complex or aggregate, and a second force, e.g., fluid force, is applied to produce force tangential to the substrate or a surface on a detection agent complex or aggregate.

In another aspect, the invention features, a method of evaluating a sample comprising red blood cells, e.g., evaluation of a sample for the presence of antibody, e.g., IgG antibody, bound to the surface of said red blood cells, said method comprising:

(a) providing a capture agent disposed on a substrate,

wherein said capture agent binds a human antibody, e.g., IgG antibody, e.g., wherein said capture agent is a AHG antibody, and wherein the substrate is configured such that cells lacking antibodies reactive with the capture agent bound to their surfaces, and thus that do not bind to the capture agent, migrate to a negative readout region (NRR);

(b) contacting said sample with the capture agent under conditions sufficient for binding, e.g., the formation of an immune complex, between the capture agent and an antibody reactive with the capture agent bound to a red blood cell, e.g., to a red blood cell surface antigen, in said sample, which binding occurs, e.g., in a positive readout region (PRR), and, e.g., sufficient to allow cells lacking antibodies reactive with the capture agent bound to their surfaces, and thus that do not bind to the capture agent to migrate to said NRR;

thereby evaluating a sample for red blood cells for the presence of antibody, e.g., IgG antibody, bound to the surface of said red blood cells.

In an embodiment, said sample comprises red blood cells that have, or may have, antibodies bound thereto and wherein said red blood cells and said antibodies are from the same person

In an embodiment, said evaluation comprises a direct agglutination test.

In an embodiment, said sample comprises plasma from a person and red blood cells that are not from said person, e.g., red cells from another person or reagent or reference red blood cells.

In an embodiment, said antibodies from said plasma are bound to said red blood cells.

In an embodiment, said evaluation comprises an indirect agglutination test.

In an embodiment, the method further comprises:

classifying the PRR as positive or non-positive for presence of cells (In an embodiment, this can be done by determining if the number of cells in the PRR meets a predetermined reference value(s)).

In an embodiment, the method further comprises:

classifying the NRR as positive or non-positive for presence of cells (In an embodiment, this can be done by determining if the number of cells in the NRR meets a predetermined reference value(s)).

In an embodiment, one or both of a PRR positive score and/or a negative NRR score is obtained and said sample is classified as having red blood cells with an antibody bound thereto.

In an embodiment, one or both of a PRR negative score and/or a NRR positive score is obtained and said sample is classified as not having red blood cells with an antibody bound thereto.

In an embodiment, one or both of a PRR negative score and/or a NRR positive score is obtained and said sample is classified as having a population of red blood cells with an antibody bound thereto and also a population of red blood cells without an antibody bound thereto.

In an embodiment, said sample comprises red cells from a subject and antibodies bound thereto from said subject.

In an embodiment, responsive to the evaluation, a patient is classified as having immune hemolytic anemia or not having immune hemolytic anemia.

In an embodiment, one or both of a PRR positive score and/or a negative NRR score is obtained and said subject is classified as having immune hemolytic anemia.

In an embodiment, one or both of a PRR negative score and/or a NRR positive score is obtained and said subject is classified as not having immune hemolytic anemia.

In an embodiment, said sample comprises a component from a first subject, e.g., a candidate recipient, and a second subject, e.g., a candidate donor.

In an embodiment, said sample comprises plasma from said first subject and red blood cells from said second subject.

In an embodiment, responsive to the evaluation, a blood product from said second subject is determined to be compatible for transfusion to said first subject.

In an embodiment, one or both of a PRR positive score and/or a negative NRR score is obtained and said patient is not approved for receiving a product comprising red blood cells from a donor.

In an embodiment, one or both of a PRR negative score and/or a NRR positive score is obtained and said patient is approved for receiving a product comprising red blood cells from a donor.

In an embodiment, responsive to the evaluation, a donor product comprising red blood cells is approved or not approved administration to said patient.

In an embodiment, one or both of a PRR positive score and/or a negative NRR score is obtained and said donor product is not approved for administration to said patient.

In an embodiment, one or both of a PRR negative score and/or a NRR positive score is obtained and said donor product is approved for administration to said patient.

In an embodiment, said antibody bound to a red blood cell, e.g., to a red blood cell surface antigen, is specific for a RBC blood antigen, e.g., for an A, B or D antigen.

In an embodiment, said antibody bound to a red blood cell, e.g., to a red blood cell surface antigen, is specific for a minor RBC blood group antigen.

In an embodiment, said antibody bound to a red blood cell, e.g., to a red blood cell surface antigen, is specific for: a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl.

In an embodiment, said capture agent comprises an antibody, e.g., an anti-human globulin (AHG).

In an embodiment, said capture agent comprises one or both of anti-C3D and anti-IgG antibodies.

In an embodiment, the sample: (1) is from a subject that has had a HTR (hemolytic transfusion reaction), (2) is from a subject that has had a DHTR (delayed hemolytic transfusion reaction), (3) is from a newborn having its RBCs coated with IgG-class antibodies from the mother, such as anti-K, anti-E, or other IgG-class antibodies to major or minor antigens which are present on the newborn's RBCs but which the mother has formed IgG-class antibodies to, (4) is from a subject that has autoantibodies which attach to it's own cells, (5) is from a subject other than one with HTR, where shortened cell life occurs due to antibodies from the subject coating transfused cells, (6) is from a subject that has drug-induced non-specific binding of antibodies to a its's RBCs, (7) is from a subject that has other non-specific binding of antibodies to a it's RBCs, or (8) is from a subject that has other causes of anti-RBC antibodies.

In an embodiment, positive and negative readout regions are imaged to provide a result.

In an embodiment, the substrate is a substantially planar substrate.

In an embodiment, the method further comprises;

(d) performing one or both of
(i) classifying the PRR as:
   single or high positive (which is indicative of a sample in which all or substantially all cells are bound by an antibody);
   mixed or low positive (which is indicative of a sample having both a population of cells bound by an antibody and a population of cells not bound by an antibody); or
   negative (which is indicative of a sample lacking cells bound by an antibody); or
(ii) classifying the NRR as:
   single or high positive (which is indicative of a sample lacking cells bound by an antibody);
   mixed or low positive (which is indicative of a sample having both a population of cells bound by an antibody and a population of cells not bound by an antibody); or
   negative (which is indicative of a sample having a population of cells bound by an antibody).

In an embodiment the method comprises:

(i) classifying a sample with a positive PRR and a negative NRR as positive (which is indicate of a sample in which all or substantially all cells are bound by an antibody);
(ii) classifying a sample with a negative PRR and a positive NRR as negative (which is indicative of a sample wherein all or substantially all cells are not bound by antibodies for which the capture agent is specific to);
(iii) classifying a sample with a positive PRR and a positive NRR as mixed field (which is indicative of a sample having both a population of cells bound by an antibody for which the capture agent is specific and a population of cells not bound by an antibody for which the capture agent is specific)

In an embodiment, the sample is reported has giving a mixed field result, indicative of having both a population of cells bound by an antibody for which the capture agent is specific and a population of cells not bound by an antibody for which the capture agent is specific.

In an embodiment, positive and negative readout regions are imaged to provide a result. In an embodiment, the image analysis can identify (a) whether there is a pellet or other concentration of cells in the negative readout region, e.g., near the center or bottom of a chamber, and (b) whether there is a preselected number cells away from negative readout region.

In an embodiment, the cells in the negative readout region can be imaged by illuminating from above with visible light and imaging from below with a camera under conditions that allow resolution of a darker region of appropriate size, e.g., several millimeters in diameter. In an embodiment cells away from the negative readout position can be imaged, e.g., with a high-resolution imaging system which can resolve individual cells and determining the number density of cells at a certain distance from the negative readout region, such as in the annulus ranging from 0.5r to 0.75r from the negative readout region center, where r is the chamber radius.

In an embodiment, the substrate is configured such that the applied acceleration allows cells having antibodies bound thereto to be distinguished from cells which do not have antibodies bound thereto. E.g., applied acceleration results in migration of cells not having antibodies bound thereto into a negative readout region. The negative readout position can, e.g., be at the bottom of a chamber (e.g., a well or a tube), or distal (relative to the direction of the applied acceleration) to a portion of the substrate to which capture agent is disposed. In an embodiment, the detection of the presence of cells without antibodies bound thereto (e.g., a negative readout) is correlated with the absence of binding between the cells (or the bound antibodies) and the capture agent. In certain embodiments, the negative readout is characterized by a button or a pellet of cells. Exemplary schematics of negative readouts are shown in FIGS. 1D and 2D as samples E and F.

In an embodiment, the substrate includes inner surface of a round-bottomed chamber and the negative readout region is, e.g., at the bottom of the round-bottom chamber.

In an embodiment, the substrate includes inner surface of a conical-bottomed chamber and the negative readout region is, e.g., at the bottom of the conical-bottom chamber.

In an embodiment, substrate is substantially planar substrate.

In an embodiment, the substrate is substantially planar and the angle between the substrate and the direction of applied force, e.g., centrifugal, gravitational, fluid magnetic, electric or fluid, force, that causes migration of detection agent, is non-orthogonal or other than 90 degrees (in the case of a centrifugally applied force, theta, the angle formed by the substrate and a line perpendicular to the direction of centrifugal force, is nonzero).

In one embodiment, the negative readout region is located on the substrate. In other embodiments, the negative readout region is not located on the substrate.

In other embodiments, the method includes comparing a value for the amount of cells present in the negative readout region with a pre-selected criterion.

In other embodiments, the method includes comparing a value for the amount of cells present in the positive readout region with a pre-selected criterion.

In an embodiment, the negative readout region is disposed in a chamber, e.g., a well or tube.

In an embodiment, the chamber is disposed on a carrier, e.g., a multi-chamber or multi-well plate, e.g., a 96 well plate.

In an embodiment, the angle between the carrier and the direction of force is non normal, e.g., between 25-5, 20-7.5, or 10 degrees.

In an embodiment, differential migration of RBCs not bound with antibody and thus not complexed with the capture agent relative to the cells complexed with antibody and thus bound to capture agent across the substrate, results in migration of unbound cells into the negative readout region.

### Applied Force and Other Embodiments

In an embodiment, migration of cells not having antibody bound thereto is effected by applying acceleration, e.g., centrifugal, fluid magnetic, electric or fluid, that causes migration of uncomplexed cells.

In certain embodiments, two different forces are applied, a first force to provide force normal to the substrate or a surface and a second force to provide force tangential to the substrate or a surface. In one embodiment, the first force, e.g., a magnetic force, is applied to produce force normal to the substrate or a surface on a detection agent complex or aggregate, and a second force, e.g., fluid force, is applied to produce force tangential to the substrate or a surface on a detection agent complex or aggregate.

### Devices

In another aspect, the invention features a device for evaluating a sample, e.g., a plasma sample, from a subject, for a mixed population of cells comprising: a channel comprising a capture agent disposed on a substrate,

wherein the capture agent binds (e.g., can from a complex, e.g., an immune complex, with) cells of the first population but not cells of the second population, e.g., it binds to a surface feature, e.g., an antigen, expressed on cells of the first population but not expressed on cells of the second population, and

wherein the substrate is configured such that cells, e.g., RBCs, that do not bind to the capture agent, are distinguishable from cells that bind to the capture agent, e.g., cells that do not bind the capture agent migrate to a negative readout region.

In an embodiment, the device comprises a second channel, the second channel comprising a second capture agent disposed on a substrate,

wherein the second capture agent binds (e.g., can from a complex, e.g., an immune complex, with) cells of one but not the other of the first population and second population, e.g., it binds to a second surface feature, e.g., a second antigen, expressed on cells of the first population but not expressed on cells of the second population, and

wherein the substrate is configured such that cells, e.g., RBCs, that do not bind to the second capture agent, are distinguishable from cells that bind to the second capture agent, e.g., cells that do not bind the second capture agent migrate to a negative readout region.

Methods of making the devices disclosed herein are also encompassed by the present invention.

Methods described herein can be applied to screening and blood typing, including, but not limited to, forward typing, minor antigen typing, and extended phenotyping.

Any of the features and embodiments described herein can be combined in any order with the described methods, and/or implemented on devices and kits described herein. In one embodiment, a forward typing method or assay is combined, e.g., on the same carrier, and/or processed simultaneously.

All publications, patent applications, patents, and other references mentioned herein are incorporated by reference in their entirety.

Other features, objects, and advantages of the invention will be apparent from the description and drawings, and from the claims.

### DESCRIPTION OF THE FIGURES

FIGS. 1A-ID illustrate side views of an embodiment of forward typing well configurations and testing, and a top view of the readout regions.

FIGS. 1E-1G show a representative panel of photographs depicting the positive and negative readouts of the forward typing assays.

FIGS. 2A-2D illustrate side views of an embodiment of extended phenotyping well configurations and testing, and a top view of the readout regions.

FIG. 3A is a schematic top plane view of a centrifuge operating in a clockwise direction.

FIGS. 3B-3C are schematic views illustrating forces as applied to objects on an incline plane disposed in an operating centrifuge.

FIG. 4 illustrates schematic and/or perspective view representations of exemplary chamber configurations.

FIG. 5 is a representative photograph of positive and negative readouts detected using antigen typing assays.

### DETAILED DESCRIPTION

Traditional agglutination tests on samples having more than one population of cells typically result in a “mixed field” reaction, wherein some cells agglutinate and others do not. Detection of mixed field reactions are important for proper patient management of the patient and to ensure subsequent testing and transfusion decisions are made correctly in light of the dual population of red cells in the patient. A mis-identification of the patient as simply being positive or negative can lead to decisions and actions which give rise to serious medical consequences.

The present invention provides, at least in part, methods, devices, kits and assays for evaluating a sample (e.g., a plasma sample, a serum sample, or a whole blood sample) from a subject for the presence of more than one population of cells, e.g., more than one population of red blood cells (RBCs). In certain embodiments, cells of the first population of cells have a surface characteristic, e.g., they present a first antigen, and cells of the second population of cells do not have the surface characteristic, e.g., they do not present the antigen. In one embodiment, the sample includes a mixed cell population, e.g., a first and second population of cells (e.g., first and second RBCs, respectively). For example, a common cause of mixed population of cells is a recent transfusion, such as an A+ subject receiving A−, O−, or O+ cells. Alternatively, a subject with a bone marrow transplant may produce two distinct populations of red blood cells. Additionally, some subjects are natural chimera with cells of two different genetic make-ups. This may occur, for example, by the fusion of embryos into a single embryo or the exchange of cells between embryos.

In other embodiments, the sample includes a mixed population of red blood cells, some of which have IgG-class antibodies present on the surface of the cells and others which do not have surface-bound IgG-class antibodies. This may occur for a variety of reasons including, but not limited to, (1) a HTR (hemolytic transfusion reaction), (2) a DHTR (delayed hemolytic transfusion reaction), (3) coating of a newborn's RBCs with IgG-class antibodies from a mother, such as anti-K, anti-E, or other IgG-class antibodies to major or minor antigens which are present on the newborn's RBCs but which the mother has formed IgG-class antibodies to, (4) autoantibodies which attach to one's own cells, (5) non-HTR cases where shortened cell life occurs due to antibodies from the patient coating transfused cells, (6) drug-induced non-specific binding of antibodies to a person's RBCs, (7) other non-specific binding of antibodies to a person's RBCs, or (8) other causes/situations.

The present invention can be applied to blood typing, including, but not limited to, forward typing, minor antigen typing, extended phenotyping and direct agglutination test (DAT).

The present invention also provides solid phase DAT and IAT.

### DEFINITIONS

Certain terms are first defined.

“Blood group,” or “blood type,” as used herein refers to any of the immunologically distinct, genetically determined classes of human blood that are based on the presence or absence of certain antigens. Blood groups are typically clinically identified by characteristic agglutination reactions. Blood group antigens which are typically associated with the ABO blood group system, and include the A, B, AB, and O blood groups.

“Blood typing,” as used herein, refers to test(s) performed in transfusion medicine, including, but not limited to, testing to detect: red cell antigens (e.g., ABO and D antigens), antibodies against red cell antigens, and/or antibodies on the surface of red cells. Blood types are typically classified as ABO Rh “blood type” commonly listed on the donor cards for blood donors (e.g., A Rh Pos, A Rh Neg, B Rh Pos, B Rh Neg, O Rh Pos, O Rh Neg, AB Rh Pos, AB Rh Neg).

“Coombs Crossmatch Test” or “Coombs Crossmatch” or “TAT Crossmatch”, as used herein refers to testing the compatibility of donor red cells and patient plasma in such a way that IgG-class antibodies in the patient blood plasma which are specific to minor antigens on the donor red blood cells or which otherwise may bind to donor red blood cells may give a positive reaction.

“Direct Agglutination Test” or “DAT,” and “Indirect Agglutination Test” or “IAT” as used herein refers to testing for detection of antibodies on the surface of cells, e.g., red blood cells. In certain embodiments, the antibodies are IgG-class antibodies. In embodiments described herein the DAT or TAT is performed on a substrate, e.g., the capture agent, e.g., an AHG antibody is disposed on, e.g., adhered to, a substrate. In embodiments the DAT is used to detect subject antibodies bound to subject red blood cells. In an embodiment the TAT is used to detect the binding of antibodies from a first subject, e.g., a patient or candidate recipient, to red blood cells from a second subject, e.g., a candidate donor of a product comprising red blood cells, e.g., blood.

“Extended phenotyping”, as used herein, refers to testing for the presence or absence of each of a collection of red cell minor blood group antigens on the surface of a sample of red blood cells. For example, an extended phenotype could test for each of D, C, c, E, e, and K. As another example, and extended phenotype could test for each of D, C, c, E, e, K, Jk^{a}, Jk^{b}, Fy^{a}, Fy^{b}, S, and s. As another example, an extended phenotype could test for each of D, C, c, E, e, K, k, Jk^{a}, Jk^{b}, Fy^{a}, Fy^{b}, M, N, S, s, Le^{a}, Le^{b}, and Pl, which may be referred to specifically as a “complete extended phenotype” or “full extended phenotype”.

“Forward typing,” as used herein, refers to determination of the A/B/O/D type by detecting the presence or absence of A, B, and D antigens on red blood cells.

“Minor antigen typing,” as used herein, refers to testing for the presence or absence of one or several specific red cell minor blood group antigens on the surface of a sample of red blood cells. For example, a minor antigen type test may test for the E antigen. As another example, one may perform minor antigen typing for both the K antigen and Jk^{a} antigen, wherein one performs a minor antigen test for each of K and Jk^{a}.

“Negative readout region,” as used herein, is a region in which a signal can indicate the absence or decrease of a surface characteristic (e.g., the absence or decrease level of an antigen), on the surface of a cell, e.g., a red blood cell. In certain embodiments, a negative readout region is detected by the behavior or the positional distribution of the cell without the surface characteristic on a substrate, e.g., a substantially planar substrate. Exemplary changes in the behavior or positional distribution of the cells without the surface characteristic can include a measure of one or more of: a change in the distribution of the cells, e.g., on the substrate; a change in the coverage of the cells, e.g., on the substrate; a change in the amount of aggregation of the cells, e.g., across the substrate (e.g., a change in the amount or distribution of complexed cells to a substrate (e.g., a functionalized substrate) relative to uncomplexed cells); a change in the number of cells, e.g., across a region of the substrate; or a change in the strength of adherence of the uncomplexed cells relative to the complexed cells (e.g., as detected by optical trapping).

“Positive readout region,” as used herein, is a region in which a signal can indicate the presence (e.g., degree) of a surface characteristic (e.g., the presence or increased level of an antigen), on the surface of a cell, e.g., a red blood cell. In certain embodiments, a positive readout region is detected by the behavior or the positional distribution of the cell with the surface characteristic on a substrate, e.g., a substantially planar substrate. Exemplary changes in the behavior or positional distribution of the cells with the surface characteristic can include a measure of one or more of: a change in the distribution of the cells, e.g., on the substrate; a change in the coverage of the cells, e.g., on the substrate; a change in the amount of aggregation of the cells, e.g., across the substrate (e.g., a change in the amount or distribution of complexed cells to a substrate (e.g., a functionalized substrate) relative to uncomplexed cells); a change in the number of cells, e.g., across a region of the substrate; or a change in the strength of adherence of the uncomplexed cells relative to the complexed cells (e.g., as detected by optical trapping).

“Readout region,” as used herein, is a region, e.g., a pre-selected region, from which a signal, e.g., a signal corresponding to the presence (e.g., degree) or absence of the surface characteristic, is collected. The readout region can be disposed on a substrate, e.g., a substantially planar substrate. In some embodiments, the readout region is disposed on a portion or the entire surface of a well, a tube, or other enclosure. In certain embodiments, the well, tube or enclosure is a round-bottom, conical, or a flat bottom well, tube or enclosure. In certain embodiments, the well, tube or enclosure has a substantially flat surface.

“Substantially planar substrate,” as used herein, means a substrate or a region of a substrate, which has one or more of the following properties:

(1) it is sufficiently planar that the desired ratio of normal force and tangent force can be maintained precisely or approximately throughout the substantially planar substrate, e.g., when accelerated with a centrifuge;

(2) the surface vector S (which is normal to the surface of the substantially planar substrate region) is constant or does not does not vary in angle, relative to its average, by more than 2, 5, or 10 degrees across the substantially planar substrate:

(3) the angle between the surface vector S (which is normal to the surface of the substantially planar substrate region) and a reference vector R, e.g., the symmetry axis of a cone, is constant, or varies by no more than 2, 5, or 10 degrees, across the substantially planar substrate substrate (thus, the surface of a perfect cone is a substantially planar substrate, as is a region of a paraboloid in the vicinity of its symmetry axis); or

(4) the ratio of the normal force to the tangent force does not exceed 110%, 130%, or 200%, or fall below 90%, 70% or 50%, of its average value within the substantially planar substrate, e.g., when accelerated with a centrifuge.

When disposed in a well, tube or other enclosure, the substantially planar substrate need not occupy the entire bottom of the enclosure. The substantially planar substrate may be continuous with other substrate regions that are not substantially planar. In an embodiment, the substantially planar substrate has a surface area of 20-200, 4-40, 0.4-10, or 0.2-10 mm². In an embodiment the substantially planar substrate is of sufficient area that it allows development of a substantial difference in migration between cells with or without a surface characteristic.

A well, tube, or other enclosure, for use in a method of device described herein can comprise one or a plurality of substantially planar substrates. For example, a well may have a lower surface comprising two angled planes which meet, forming a “V”-shaped trough, and still be considered a substantially planar surface. Substantially planar substrates can be disposed on the same, or different substrates. In an embodiment having a plurality of substantially planar substrates in one well tube or other enclosure, the surface area of the plurality is 20-200, 4-40, 0.4-10, or 0.2-10 mm².

Substantially planar does not require a smooth surface. In embodiments, substantially planar substrate can have surface texturing, e.g., it can be grooved or have a roughened or dimpled surface. In an embodiment, the average displacement between the lowest and highest points of the features is less than 10 microns, 1-100 microns, or 10-50 microns. For the preceding determination of the surface vector in cases where the surface has structure or texture on length scales smaller than 5, 10, 25, or 50 microns, the surface vector is taken to be the vector normal to the “average surface” at that point, where the average surface is calculated by fitting the neighborhood of size 5, 10, 25, or 50 microns to a plane. In one embodiment, the surface of the substantially planar substrate resides substantially in a plane.

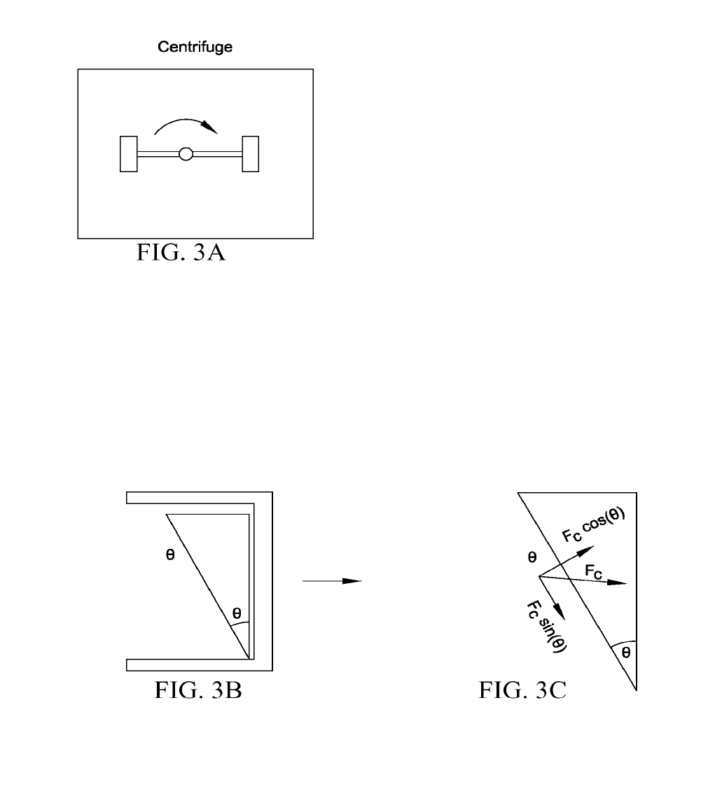

The substantially planar substrate can be disposed on a substrate which comprises a substantially planar region or substrate and a region which is not substantially planar substrate. A region which is not a substantially planar substrate could be: (a) a “capture feature” for capturing cells as they travel across the surface, which, in embodiments, optimizes the detection, e.g., optical detection, of unbound cells, (b) an “aggregate nucleation region” which, in embodiments is steeper than the substantially planar region and, relative to the direction traverse of cells across the substantially planar substrate, is upstream of it, which, in embodiments facilitates formation of aggregates, e.g., small aggregates, to more quickly clear off the substantially planar region for negative samples, and (c) a negative readout region which may or may not be part of the substantially planar region, and which, in embodiments, is where aggregates, e.g., large aggregates, to traverse to. FIGS. 3B-3C show exemplary substantially planar substrates.

As used herein, a population of cells is substantially devoid of a surface feature or characteristic, e.g., a cell surface antigen, if the average number of the surface feature, e.g., cell surface antigen, molecules on a cell of the population is less than 5, 1, 0.1 or 0.01% of the average number of surface feature, e.g., cell surface antigen, molecules on a cell of the population which possess or expresses the cell surface feature and to which it is being compared. E.g., a RBC that does not express A antigen and a RBC that does express A antigen. Typically, the number of cell surface features on cells that are essentially devoid of the cell surface feature is such that the assay shows no detectable binding to the capture reagent under the conditions in which the assay is used.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

### Forward Typing, Minor Antigen Typing, Extended Phenotyping, Direct Agglutination Test (DAT), Indirect Agglutination Test (IAT) and Coombs Crossmatch Test

In one aspect, the invention provides methods, devices and kits for evaluating a sample for a red blood cell antigen, e.g., forward typing, minor antigen typing, extended phenotyping, Direct Agglutination Test (DAT), Indirect Agglutination Test (IAT) or Coombs Crossmatch Test. In embodiments the method includes:

(a) contacting a capture agent which binds to a first population of cells, e.g., binds to a surface feature on the cell (e.g., a red blood cell), said capture agent being disposed on a surface (e.g., a functionalized surface as described herein) with the sample, e.g., a sample containing one or more different population of red blood cells (e.g., first and second rbc's as described herein), under conditions sufficient for the formation of a complex between said capture agent and a red blood cell in said sample to occur, wherein said surface feature is an antigen on the cell, e.g., a red blood cell antigen (referred to herein as “complexed cells”);

(b) separating the complexed cells, e.g., by causing differential migration of the complexed cells relative to a second population of cells (e.g., red blood cells without the surface feature, and thus not complexed with said capture agent (“uncomplexed cells”)), across said substrate.

A change, e.g., an increase or decrease, in the amount of complexed and/or uncomplexed cells, e.g., red blood cells, is correlated with the amount of said surface feature, e.g., red blood cell antigen, in said sample, thereby evaluating the sample for cells with or without the surface feature. Capture agents can be immobilized on a substrate or surface (i.e., solid phase) thereby being able to capture the surface feature, e.g., form a complex with a cell or cell population displaying the surface feature.

In one embodiment, the surface feature is an antibody bound to a red blood cell. In certain embodiments, the antibody bound to the red blood cell is an IgG-class antibody. In such embodiments, the complexed cells are bound by an antibody, e.g., an anti-IgG antibody, immobilized to a substrate, e.g., a substantially planar substrate. In some embodiments, a sample includes a mixed population of red blood cells, some of which have IgG-class antibodies present on the surface of the cells and others which do not have surface-bound IgG-class antibodies.

In other embodiments, the capture agent is a red blood cell antigen binding agent, e.g., anti-red blood cell antigen antibody. The surface feature, e.g., the red blood cell antigen, can be a blood-type antigen, e.g., an A, B, AB or D antigen. In one embodiment, the method is a forward typing method, e.g., comprises the detection of a red blood cell antigen chosen from an A, B, or D antigen.

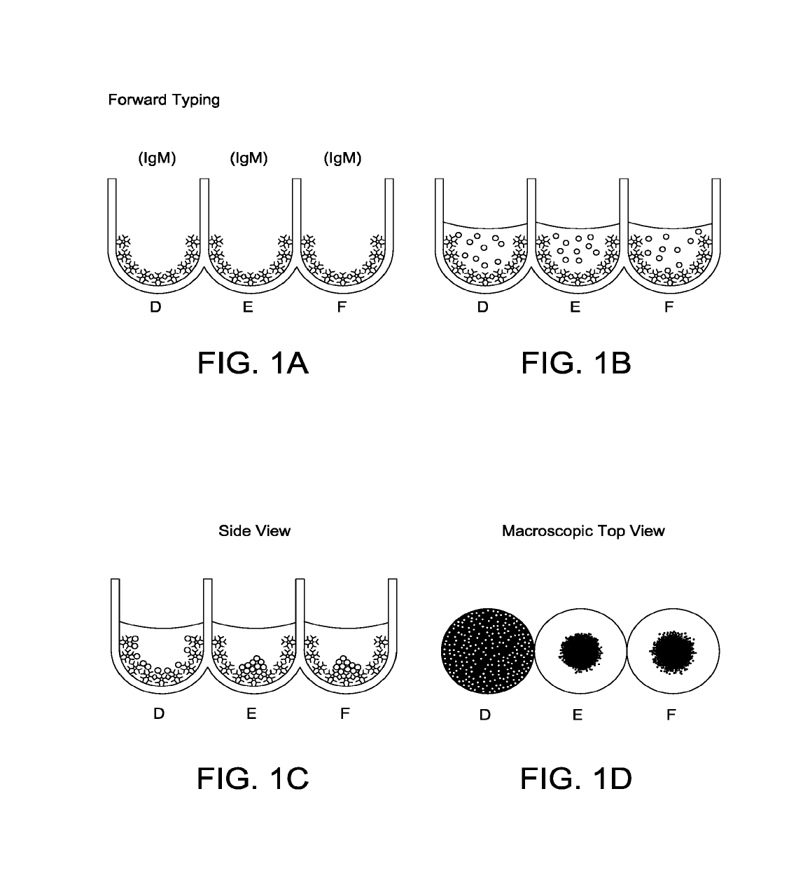

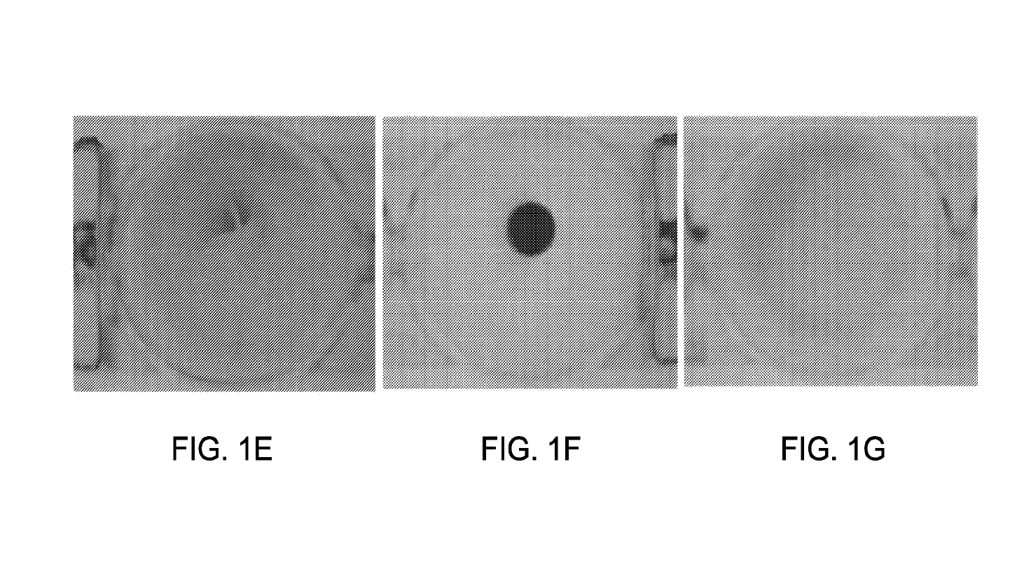

An embodiment of a forward typing assay is depicted in FIGS. 1A-1D. Referring to FIG. 1A, a side view of three forward typing, U-shaped wells labeled D, E, and F is depicted. Each well is modified to contain a red blood cell binding agent disposed on (e.g., covalently or non-covalently bound to) its inner surface. In one embodiment, the red blood cell binding agent is an anti-red blood cell antigen antibody (e.g., an IgG or an IgM (as shown), or a combination thereof). In other embodiments, the red blood cell antigen binding agent can be a molecule that binds to a red blood cell antigen, e.g., a protein, a peptide or a carbohydrate. In other embodiments, the red blood cell antigen binding agent is a plant-derived binding agent, e.g., a lectin. In the embodiments shown in FIGS. 1A-1D, each well contains a different IgM antibody. A sample, e.g., plasma, serum or whole blood sample containing red blood cells (depicted as open circles in FIG. 1B), is added under conditions sufficient for the formation of a complex between said red blood cell antigen binding agent, e.g., anti-red blood cell antigen antibody, and a red blood cell in said sample to occur (referred to herein as “complexed cells”). In certain embodiments, the complexed cells are separated from the uncomplexed cells, e.g., by causing differential migration of red blood cells not complexed with said red blood cell antigen binding agent, e.g., anti-red blood cell antigen antibody (“uncomplexed cells”), relative to the complexed cells, across said substrate. The formation of complexed cells is represented well D in FIG. 1C. The positive readout is represented as a uniform distribution of the complexed cells across the inner surface of the well, represented in FIG. 1D as a homogeneous distribution across the entire top view of well D. Negative readouts are shown in a side view of wells E and F, depicted as an aggregate of uncomplexed cells. A top view of the negative readout is shown in schematic form in FIG. 1D, where the aggregated, uncomplexed cells are clustered in the center portion of the wells. FIGS. 1E and 1G show representative positive readouts, and FIG. 1F shows a representative negative readout for the forward typing assays described herein.

Detection of a mixed field in a forward-type assay is typically presented as a mixture of an aggregate of uncomplexed cells and a relative homogeneous distribution of the complexed cells across the substrate (not shown). In exemplary embodiments, it can be visualized as a mixture of a positive and a negative readout described above.

In other embodiments, the surface feature, e.g., red blood cell antigen, is chosen from at least 1, 2, 3, 4, 5, 6, 7, 8, 9, 10, 11, 12 or all of the RBC antigens provided in Table 1. In one embodiment, the red blood cell antigen is a minor antigen. In one embodiment, the red blood cell antigen is chosen from one, two, three, four, five, six, seven, eight, nine, ten, eleven, twelve, or more, or all of: a Rhesus antigen, e.g., one or more or all of D, C, c, E, or e; an MNS antigen, e.g., one or more or all of M, N, S, or s; a Kidd antigen, e.g., one or both of Jk^{a} or Jk^{b}; a Duffy antigen, e.g., one or both of Fy^{a} or Fy^{b}; a Kell antigen, e.g., one or both of K or k; a Lewis antigen, e.g., one or both of Le^{a} or Le^{b}; or a P antigen, e.g., Pl. In certain embodiments, the red blood cell antigen analyzed includes at least the following RBC antigens: (1) D, C, E, e, c, and K; (2) D, C, E, e, c, K, Fy^{a} and Jk^{a}; or (3) D, C, E, e, c, K, Fy^{a}, Fy^{b}, Jk^{a}, Jk^{b} S, and s.

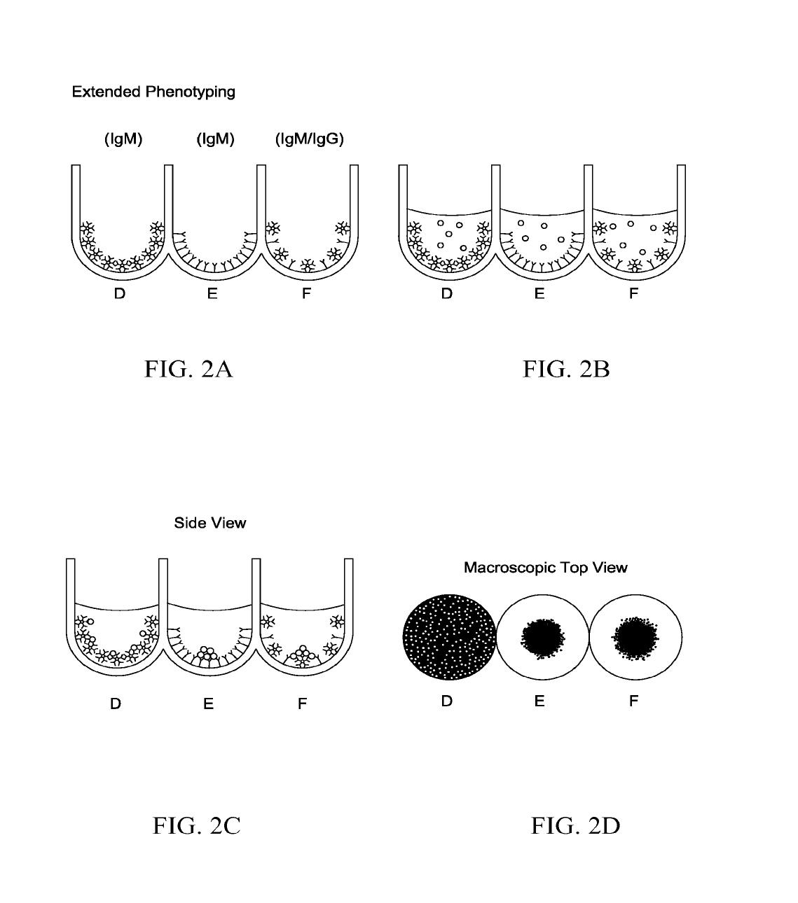

FIGS. 2A-2D provide a schematic of one embodiment of extended phenotyping assays. Similar to the forward typing assays, three wells, labeled D, E and F, are depicted, each one containing a different red blood cell antigen binding agent. The red blood cell antigen binding agent can be any molecule that binds to a red blood cell antigen, e.g., a protein, a peptide or a carbohydrate. In one embodiment, the red blood cell antigen binding agent is an anti-red blood cell antigen antibody (e.g., an IgG or an IgM, or a combination thereof). In other embodiments, the red blood cell antigen binding agent is a plant-derived binding agent. In the embodiments shown in FIGS. 2A-2D, each well contains a different IgM antibody (depicted as a pentameric structure in well D), IgG antibody (depicted as a “Y” in well E), or a combination of IgM and IgG antibodies (in well F), e.g., disposed on the inner, lower portion of the well. A sample, e.g., plasma, serum or whole blood sample containing red blood cells (depicted as open circles in FIG. 2B), is added under conditions sufficient for the formation of a complex between said red blood cell antigen binding agent, e.g., anti-red blood cell antigen antibody, and a red blood cell in said sample to occur (referred to herein as “complexed cells”). In certain embodiments, the complexed cells are separated from the uncomplexed cells, e.g., by causing differential migration of red blood cells not complexed with said red blood cell antigen binding agent, e.g., anti-red blood cell antigen antibody (“uncomplexed cells”), relative to the complexed cells, across said substrate. The formation of complexed cells is represented well D in FIG. 2C. The positive readout is represented as a uniform distribution of the complexed cells across the inner surface of the well, represented in FIG. 2D as a homogeneous distribution across the entire top view of well D. Negative readouts are shown in a side view of wells E and F, depicted as an aggregate of uncomplexed cells. A top view of the negative readout is shown in schematic form in FIG. 2D, where the aggregated, uncomplexed cells are clustered in the center portion of the wells.

Detection of a mixed field in an extended phenotyping assay is typically presented as a mixture of an aggregate of uncomplexed cells and a relative homogeneous distribution of the complexed cells across the substrate (not shown). In exemplary embodiments, it can be visualized as a mixture of a positive and a negative readout described above.

In an embodiment, the change, e.g., presence or absence, of detection uncomplexed cells is detected by in one or more of: a difference in the amount of the uncomplexed cells relative to the complexed cells (which remain substantially bound to the substrate); a difference in the distribution of the uncomplexed cells relative to the complexed cells (which remain substantially bound to the substrate), e.g., on a substrate; a difference in the amount of aggregation of the uncomplexed cells relative to the complexed cells; or a difference in the strength of adherence of the uncomplexed cells relative to the complexed cells (e.g., as detected by optical trapping).

In one embodiment, the separation is effected by applying acceleration, e.g., centrifugal, fluid magnetic, electric or fluid, that causes migration of the complexed and uncomplexed cells.

In an embodiment, the surface is configured such that the applied acceleration results in migration of uncomplexed cells into a complex, e.g., at the bottom of a chamber (e.g., a well or a tube). In an embodiment, the detection of the presence of uncomplexed cells (e.g., a negative readout) is correlated with the absence of said anti-RBC antigen antibody and said sample. In certain embodiments, the negative readout is a button or a pellet. Exemplary schematics of negative readouts are shown in FIGS. 1D and 2D as samples E and F.

In one embodiment, the detection of the presence of complexed cells (e.g., a positive readout) is correlated with the presence of binding between said anti-RBC antigen antibody and said sample. In certain embodiments, the positive readout is detected as a haze. A schematic of the top views of the readout in chamber is depicted in FIGS. 1D and 2D, where a positive readout is detected as a haze in sample D of FIGS. 1D and 2D.

In an embodiment, the readout region is disposed in a chamber, e.g., a well, tube, or other enclosure. In some embodiments, the readout region is disposed on a portion or the entire surface of a well, a tube, or other enclosure. In certain embodiments, the well, tube or enclosure is a round-bottom, conical, or a flat bottom well, tube or enclosure. In certain embodiments, the well, tube or enclosure has a substantially flat surface.

In an embodiment, the chamber is disposed on a carrier, e.g., a multi-chamber or multi-well plate, e.g., a 96 well plate.

In other embodiments, the capture agent is disposed on a substantially planar substrate, and the angle between said substantially planar substrate and the direction of applied force, e.g., centrifugal, gravitational, fluid magnetic, electric or fluid, force, that causes migration of detection reagent, is non-orthogonal or other than 90 degrees (theta, the angle formed by the substantially planar substrate and a line perpendicular to the direction of centrifugal force, is nonzero).

In an embodiment, the angle between said substrate and the direction of force is non normal, e.g., between 25-5, 20-7.5, or 10 degrees.

In other embodiments, a force is applied, e.g., an acceleration (e.g., centrifugal acceleration) is applied, at an angle such that the cells that do not bind to said capture agent migrate across said substrate or surface, e.g., substantially planar substrate or surface.

In one embodiment, the negative readout region is located on said substrate, e.g., substantially planar substrate. In other embodiments, the negative readout region is not located on said substrate, e.g., substantially planar substrate.

In yet other embodiments, the method or device further includes detecting the presence of said cells in a first positive readout region, e.g., on said substrate, e.g., substantially planar substrate.

In other embodiments, the method or device further includes comparing a value for the amount of cells present in said first positive readout region with a pre-selected criterion, and if said value meets said pre-selected criterion classifying said sample, e.g., as positive.

In other embodiments, the method or device further includes detecting the presence of said cells in said first negative region.

In other embodiments, the method or device further includes comparing a value for the amount of cells in said first negative readout region with a pre-selected criterion, and if said value meets said pre-selected criterion classifying said sample, e.g., as negative.

### Exemplary Configurations and Applied Forces for Solid-Phase Capturing Methods

In blood typing assays a force can be applied in order to produce a more clear measurement. For instance, if a round or ‘U’ bottom well is used in conjunction with a swinging bucket centrifuge, unbound cells will be forced to the bottommost portion of the well. If a specific bond is formed between a cell and the capture agent immobilized to the solid phase, and the centrifugal force is less than the binding force existing between the cell bound to the solid-phase, the cell will remain (to some degree) dispersed across the solid-phase. If no binding occurs or binding is insufficient to counter the centrifugal force, the cells will migrate to the lowest position in the well. Hence, if a ‘button’ of cells is present at the bottommost portion of the well, a negative result can be inferred. If such a ‘button’ is absent (or diminished) a positive result can be inferred. For mixed field, a button and dispersed distribution of cells is expected. Thus, in one embodiment the test result is determined using scores for both the negative readout region and the positive readout region.

Conventional assays that utilize round bottom or non-planar geometries do not produce optimal sensitivity. To begin, the geometry of the well and the configuration of the centrifuge control the magnitude and the direction of the forces applied to the cells. For instance, if a flat-bottom surface is used in conjunction with a swinging bucket centrifuge wherein the acceleration is strictly normal to the surface, a cell will only experience a normal force that drives it to the surface and no differentiation between bound an unbound can be made. Alternatively, if an inclined plane is introduced such that the plate resides at a non-orthogonal angle relative to the radial direction, a tangential force will be applied to the cells. The magnitude of the relevant forces in the directions tangent and normal to the inclined plane are given by the product of the centrifugal force (Fc) times the sin (tangential) or cos (normal) of the inclined plane angle. These configurations are represented in schematic form in FIGS. 3A-3C. FIG. 3A is a schematic top plane view of a swinging bucket centrifuge operating in a clockwise direction. The arrow indicates the axis of rotation. FIGS. 3B-3C is a free body diagram representing the normal and tangential forces acting on a cell (Fc is a centrifugal force). In this context, if no other limiting factors (for instance, non-specific binding) are significant in magnitude, a small theta combined with a minimal centrifugal force is ideal. Such a situation would first drive and then push an indicator into close proximity with the surface of interest. A small lateral force would push the cells across the surface at a rate dependant on the particle size, solute viscosity, and centrifugal force. In this regime (low angle and low centrifugal force) the cells would slowly travel across the solid-phase and probe potential binding sites. The low speed (as compared to those induced by high centrifugal forces or angles) increases the interaction time existing between a cell and potential binding sites. Ultimately, this increase in time should lead to a higher percentage of cells bound to the surface.

Moreover, the low centrifugal force and low angle produce less tension on a specific bond once it is formed. This leads to a greater percentage of cells bound to the surface once a measurement is concluded.

Non-planar geometries are now compared to the proposed optimal geometry. A round bottom or ‘U’ bottom well of typical design produces low angle inclines only at the very bottom of the well. Hence, most of the well area produces relatively large tangential forces and relatively weak normal forces. In addition, the bottom portion of the well that may produce optimal binding conditions is typically occupied by truly unbound cells and therefore is inaccessible to measurement. This is quite non-ideal and can be significantly improved upon by utilizing a planar geometry combined with a small incline. Nonetheless, for highly robust tests a round bottom or ‘U’ bottom well may be made to work adequately, as discussed above and in the figures.

### Exemplary Solid Phase Configurations

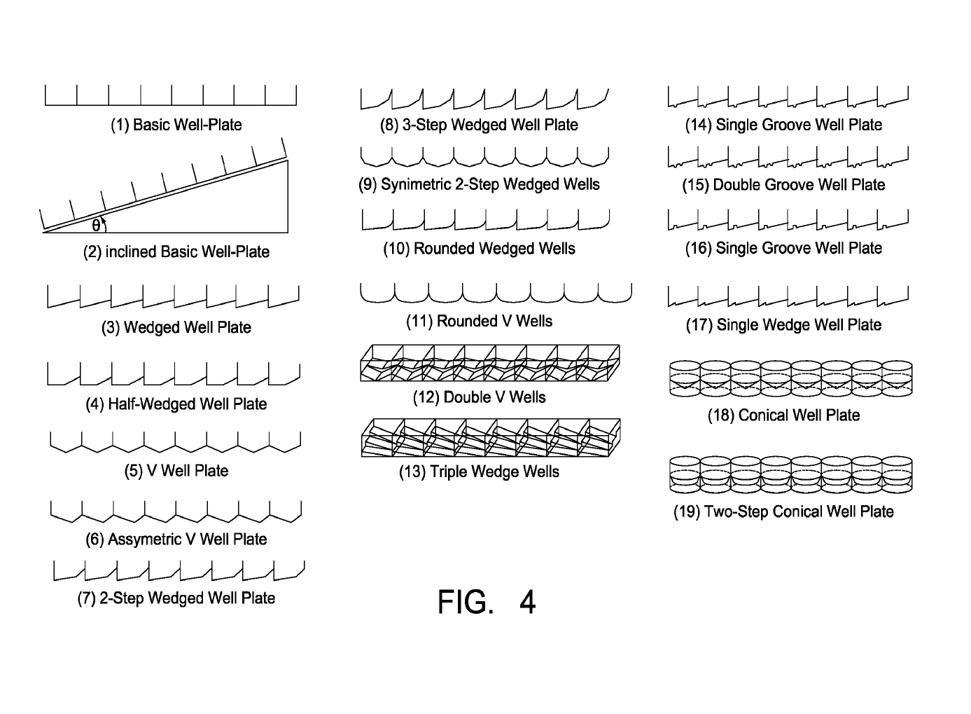

In other embodiments, the methods and devices of the invention can be carried out using one or more of the exemplary well plate configurations depicted in FIG. 4. Such plate geometries are believed to create the right balance of normal force and tangential force; to have different normal forces and tangential forces in different wells; to eliminate the radius arm problem and tilted plate problem (i.e., ensure two wells have identical force profiles even though they are in different locations, such as distance from the central rotational axis, when being centrifuged); to do different tests in different wells at the same time; to accelerate (nucleate) the avalanche effect; to do multiple tests within a single well on a single sample; or to generally improve the imaging and/or discernment of positive vs. negative samples. (Note that in many cases, it may make imaging more difficult, unless a larger depth of focus is employed or an imaging system which can scan or image out of plane is used.)

The 19 well plate geometries shown in FIG. 4 are described as follows:

(1) Basic commercial well plate.

(2) Basic commercial well-plate inclined at angle theta to produce correct ratio of F_normal to F_tangent.

(3) Custom well plate designed to have angle introduced into well bottom (manufactured into plate) rather than to be centrifuged at an angle. Note: Each well could have a different angle if required, such as for different tests. This would function similarly to (2) which we are doing now. It requires a custom well plate and a suitable reader, but eliminates the complexity of centrifuging with an angle. It also allows for different wells to be under different conditions, which is not presently possible with (2).

(4) This plate can be operated in either of two ways: (a) It can be placed into an angled centrifuge to have two angles in a given well. This may have the benefit of having a region of higher angle where the RBCs more quickly move and form aggregates of a certain size, if free. This may speed up the aggregate formation and avalanche for negative samples. The remainder of the well is at a normal, lower angle. (b) It can be centrifuged without an angle so as to have the clusters stop at a place away from the wall for easier reading.

(5) V Well Plate: This can be centrifuged without an angle. It has the benefit that unbound aggregates go to the centerline which is easier to see and measure.

(6) Asymmetric V Well Plate: As with (5) above, except the asymmetry can be used to either measure the response at two angles or to allow one to perform the test using the shallow slope side, giving more distance and area to work with.

(7) 2-Step-Wedged Well Plate: This is similar to (4) above but doesn't need to be centrifuged at an angle to obtain the two non-zero angle case. The idea is that cells first sprinkle down uniformly. Then cells quickly slide down the steeper slope and form some smallish aggregates (if they do not bind to surface). When they reach the lower slope, for negative samples, the aggregates start with significant size meaning that negative samples will have a much stronger avalanche, faster. If it is positive, the avalanche will not be initiated. Thus, this is meant as a means to nucleate and speed up the avalanching process for negative samples.

(8) 3-Step-Wedged Well Plate: This is like the 2-step-wedged well plate, except it has another wedge. The steepest wedge quickly nucleates small clusters. The next wedge allows these to grow to a certain size if the sample is negative. The main wedge allows them to grow to the “unstable” size if still no binding occurs.

(9) Asymmetric 2-Step Wedge: This is like (8) except it is symmetric so that the negative region is more easily read, since it is in the centerline.

(10) Rounded Wedged Wells Plate: The rounding at one edge achieves something equivalent to the 3-step wedged well plate.

(11) This combines the rounded wedged well plate simplicity with the convenience of symmetry so that cells collect in the centerline for negative samples, for easy and accurate reading. (Note that these rounded wells are quite distinct from what some others use at present, since most of the well is still essentially planar and at a prescribed (low) range of angles. The cells that collect do not obscure the important angle region).

(12) Double V Well Plate: This allows each sample to simultaneously be tested under two angles. This may be used for quantization, among other things.

(13) Triple Wedge Well Plate: As with the double-V well plate, this allows one to perform multiple tests on the same blood in one well. It can be used to get data at different angles, such as for quantization.

(14) Single Groove Well Plate: This has a single groove in the surface which catches unbound cells or aggregates (perhaps of a certain size or less). This makes it easier to read negative samples.

(15) Double Groove Well Plate: This has two grooves (or more) of the same or differing sizes or shapes. This can help discriminate a low level of aggregation and a higher level of aggregation.

(16) Single Hump Well Plate: This is functionally fairly similar to the Single Groove Well Plate.

(17) Single Wedge-Hump Well Plate: This is functionally pretty similar to the Single Hump Well Plate.

(18) Conical Well Plate: This is like a V Well Plate but has azimuthal symmetry.

(19) Two-Step Conical Well Plate: This is like a Symmetric Two-Step V Well Plate (#9) but has azimuthal symmetry.

### Exemplary Substrate Configurations

Methods and devices of the invention use substrates, e.g., substantially planar substrates, on which a variety of entities are disposed including, for example, capture reagents, e.g., antibodies. In certain embodiments, cells migrate across said surfaces. Numerous approaches for optimizing one or more of these behaviors are disclosed herein.

Any of the methods or devices described herein can incorporate one or more of the following features:

In one embodiment, the substantially planar substrate is adjacent a region having a steeper angles, e.g., an angle which minimizes binding of cells, aggregates.

In another embodiment, the substantially planar substrate is adjacent a region having a different surface treatment.

In an embodiment, the substantially planar substrate is adjacent a region having is configured so as to enhance nucleation, e.g., a region which increases the concentration of particles in the direction of migration.

In an embodiment, the substantially planar substrate is adjacent a region having which concentrations migrating particles, e.g., a region configured as a funnel.

In an embodiment, the substantially planar substrate is adjacent a region having a feature which improves the detectability of negative samples, e.g., a feature which impedes the passage, captures, or concentrates migrating entities, e.g., cells, aggregates. By way of example, the region can comprise a depression, e.g., a pit or groove, or an elevation, e.g., a bump or ridge, or a discontinuity or interface, e.g., between two regions.

In an embodiment the substrate comprises an interface between two surface regions wherein migrating cells, aggregates, can collect, and the presence, absence, or quantity of cells in this region can inform a test result.

In an embodiment, a substrate comprises a plurality of surfaces, e.g., planar or substantially planar in sufficient proximity allow performance of a plurality of tests, e.g., two tests, with different properties, e.g., sensitivities, e.g., to quantitate the test result.

In one embodiment, a substantially planar surface region is azimuthally symmetric.

In another embodiment, a carrier, e.g., a plate, having a plurality of substantially planar surface regions, disposed at more than 1 different angle, so that tests can be performed at different conditions, such as for doing two different tests with different parameters at the same time on the same plate.

### EXAMPLES

The invention now being generally described, it will be more readily understood by reference to the following examples, which are included merely for purposes of illustration of certain aspects and embodiments of the present invention, and are not intended to limit the invention.

### Example 1

### Forward Typing

### Materials:

The following available materials are used in this example:
(i) Anti-A, anti-B, anti-D—material from cell lines LA2, LB2, LDM1 obtained from Alba Biosciences—purified to greater than 90% and concentrated to 1 mg/mL
(ii) Round bottom microtiter plate (767-070) obtained from Greiner BioOne
(iii) Blood bank saline (C) obtained from Fisher Scientific
(iv) BSA (A7906) obtained from Sigma-Aldrich
(v) BupH PBS (28372) obtained from Pierce
(vi) Poly-1-lysine (P1524) obtained from Sigma-Aldrich
(vii) Tween 20 (P9416) obtained from Sigma-Aldrich
(viii) 96 well microtiter plate lid (656-170) obtained from Greiner BioOne

### Preparation of Antibody Solutions:

20 mL of BupH PBS is added to each of three tubes and the tubes are marked as “LA2”, “LB2”, and “LDM1”. Each tube receives 80 μL of purified anti-A (LA2), anti-B (LB2) or anti-D (LDM1) (each antibody into respectively marked tube) and the tubes are thoroughly mixed.

### Preparation of Test Surface:

Greiner medium binding round bottom 96 well plates are loaded with 100 uL of the appropriate antibody solution. The plate is covered with a lid and stored in a refrigerator at 4 C overnight. The next morning, the plate is then washed at least six times with 200 uL of saline to remove unbound protein.

### Plate Blocking:

The wells are then aspirated and 200 uL of a blocking solution (3% BSA, 0.1% Tween 20 in BupH PBS) is added to each. This is repeated for all rows. The plate is then covered with a lid and stored at 4 C for 36 hours. After this time has elapsed, the plate is ready for use.

### Preparation of Red Cells:

Red blood cells are diluted into 0.9% saline to a final concentration of 0.04% (i.e. first 10 uL packed RBCs mixed with 90 uL 0.9% saline and 4 uL of this dilution is mixed with 1000 uL 0.9% saline).

### Test Procedure:

100 uL of the 0.04% RBC solution is added to one well of LA2,1 LB2 and LDM1. The strip (being held by the 96-well plate frame) is placed into a swinging bucket centrifuge and spun for 1.5 minutes at 200 g's and an additional 1.5 minutes at 500 g's.

### Result Interpretation:

The plate is then examined for binding—a negative binding event is designated as the formation of a red cell button in the well; a positive binding event is designated as the lack of a red cell button (there is a red “haze” present from the red cells binding over the surface of the well); a mixed field result exhibits both a button near the bottom of the well as well as a red “haze” or significant presence of red cells binding over the surface of the well. A mixed field result can be identified as satisfying the detection threshold for a button as well as for the “haze” or presence of cells away from the expected button. Alternately, a mixed field result can be identified by scoring test results in both the positive readout region and the negative readout region and using both of these scores to determine if the result is mixed field.

FIGS. 1E-1G display a typical result of this assay. FIG. 1E is an image of a well coated with anti-A as described herein, exposed to sample, and then centrifuged. FIG. 1E shows a ‘haze’ of blood cells indicating that binding between the cells and the surface is present and that the cells in the sample present the A antigen. FIG. 1F is an image of well coated with anti-B as described herein, exposed to sample, and then centrifuged. The figure shows a pellet of red blood cells indicating that binding between the cells and the surface is absent. Thus, the cells in the sample do not present the B antigen. FIG. 1G is an image of the well coated with anti-D as described herein, exposed to sample, and then centrifuged. The figure shows a ‘haze’ of blood cells indicating that binding between the cell and the surface is present. Thus, the cells contained in the sample present the D antigen. Therefore, the blood type of this particular sample may be interpreted as A+.

### Example 2

### Minor Antigen Typing and Extended Phenotyping

### Materials:

(i) Protein L from Peptostreptococcus magnus (P3101) obtained from Sigma Aldrich

(ii) Anti-D (Z031) obtained from Alba Bioscience

(iii) Anti-c (Z083) obtained from Alba Bioscience

(iv) Anti-C (Z063) obtained from Alba Bioscience

(v) Anti-e (Z094A) obtained from Alba Bioscience

(vi) Anti-E (Z073) obtained from Alba Bioscience

(vii) Anti-Jka (Z162) obtained from Alba Bioscience

(viii) BupH phosphate buffered saline obtained from Pierce

(ix) Blood bank saline (22-026-401) obtained from Fisher Scientific

(x) Round bottom 96 well plates (767-070) obtained from Greiner BioOne

(xi) 96 well plate lids (656-170) obtained from Greiner BioOne

### Plate Preparation:

Protein L was dissolved in PBS at a concentration of 1 mg/mL. It was then diluted 5000-fold with PBS and 75 uL of this solution was pipetted into each well. The plate was covered with a lid and allowed to incubate overnight at 4 C. After incubation was complete, each well was washed with 200 uL of saline 5× and then 75 uL of the desired antibody (anti-D, anti-c, anti-C, anti-e, anti-E, anti-Jka) was added to each well and the reaction allowed to proceed for 4 hours at room temperature. The wells were once again washed with 200 uL of saline 4×.

### Test Procedure:

100 uL of a 0.04% suspension of test red blood cells are added to each well. The plate is centrifuged at 50 g's for 8 mins in a swinging bucket centrifuge and then the plate is read. A tightly packed pellet at the bottom of the well indicates that the sample is negative for the antigen in question, and a dispersed or ‘hazy’ layer of test cells indicates a positive.

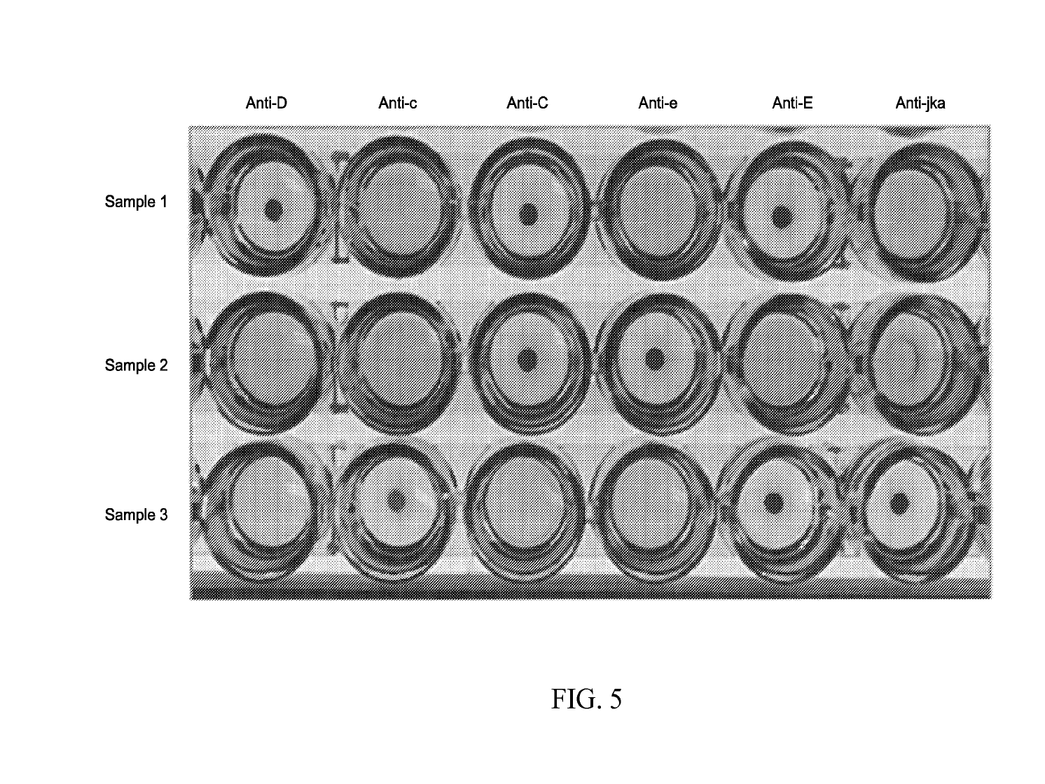

FIG. 5 is an image of three samples tested using the assay described herein. Each row represents one distinct sample. Each column represents one distinct specificity (D, c, C, e, E, Jk^{a}). The figure shows that Sample 1 has the following antigen profile D−, c+, C−, e+, E−, Jka+. Sample 2 has the following antigen profile: D+, c+, C−, e−, E+, Jka+. Sample 3 has the following antigen profile: D+, c−, C+, e+, E−, Jka−.

### Example 3

### Antigen-Typing with Mixed-Field Detection Capability

### Background

Blood samples from some patients may present different populations of cells with different antigen expression. A common cause of this is a recent transfusion. For example, an A+ subject may receive A−, O−, or O+ red cells. Alternatively, a subject with a bone marrow transplant may produce two distinct populations of red blood cells. Additionally, some subjects are natural chimera with cells of two different genetic make-ups. This may occur, for example, by the fusion of embryos into a single embryo or the exchange of cells between embryos.

Traditional agglutination tests on such samples result in a “mixed field” reaction, wherein some cells agglutinate and others do not. Mixed field reactions can be readily observed by a careful expert blood banker. Detection of mixed field reactions are important for proper patient management, and to ensure subsequent testing and transfusion decisions are made correctly in light of the dual population of red cells in the patient. A mis-identification of the patient as simply being positive or negative can lead to decisions and actions which can give rise to serious medical consequences.

Current instruments for blood type testing relying on automation of traditional hemagglutination do not have the capability of detecting mixed field reactions. These instruments may report negative, weakly positive, or positive results depending on the relative sizes of the two cell populations.

### Experimental Preparation and Test:

Methods and devices for performing a solid phase major or minor antigen test wherein the result may be a positive, a negative, or a mixed field are described herein. The surface of a conical, round-bottom, flat-bottom, substantially planar, or similar well is coated with a capture agent (e.g., antibodies) as described, for example, in Example 1: Forward Typing and Example 2: Minor Antigen Typing and Extended Phenotyping.

Whole blood to be tested is diluted (0.04% dilution, as described in the above-Examples).

A volume of diluted red cells is introduced. For example, per the above-examples, 100 uL of a 0.04% RBC solution can be used. More optimal results may be obtained by using a lower total volume of cells, such as 1 uL to 100 uL of a 0.04% RBC solution. The lower volume of cells reduces interactions between stationary bound cells and moving unbound cells, improving the ability of unbound cells to migrate to the bottom of the well and reducing the chances that moving unbound cells may collide with bound cells and cause bound cells to become unbound. One skilled in the art can best optimize the volume of cells used through testing procedures known in the art.

The cells are centrifuged to the surface, as described in the above-Examples. Note that added centrifugation may optionally be used to better reveal samples wherein most cells bind to the surface and a minority of cells are not bound. This can be achieved by extending the centrifugation speed and/or duration, or by adding another centrifugation step which is at higher speed. For example, a final 2-minute spin at 700 g can be added for forward typing, or a final 100 g spin for 5 minutes can be added for minor antigen typing.

Cells presenting the antigen are expected to bind to the antibody-covered surface. Cells not presenting the antigen for which the surface antibody is specific will not bind to the surface. The low overall coverage of the surface can enable most unbound cells to move to the lowest point, as the bound cells do not form a tight carpet and will generally not strongly impede the migration of unbound cells down the surface.

The test results can be determined by imaging the wells. Specifically, the image analysis software can be used to identify (a) whether there is a pellet of cells near the center of the well, and (b) whether there is a sufficient number cells away from the center of the well, above the background level that may occur in a negative sample through non-specific binding or other non-specific effects. The pellet can be readily imaged, such as by illuminating from above with visible light and imaging from below with a camera, looking for a darker region of appropriate size, such as 0.5 millimeters, 1.0 millimeters or 2.0 millimeters. Imaging cells away from the center can be done, for example, by having a high-resolution imaging system which can resolve individual cells, and determining the number density of cells at a certain distance from the well center, such as in the annulus ranging from 0.5r to 0.75r from the center, where r is the well radius. A threshold cell concentration is selected by testing “mixed field” samples. Alternately, determination of whether there is a threshold concentration of cells may be made through imaging or non-imaging detection which does not resolve individual cells. The results are interpreted as follows:

Pellet and very few or no cells on angled surfaces, indicating that the red blood cells are negative for antigen;

Many cells on angled surfaces and no detectable pellet, indicating that the red blood cells positive for the antigen;

Both pellet and many cells on angled surfaces, indicating a mixed field, e.g., having two populations of red blood cells, one positive and another one negative for the antigen; or

If no cells are detected anywhere, it may indicate an error in, e.g., pipetting sample or centrifuging, or other error.

Described herein is a solid phase forward typing test, which is already unusual. General methods used include: (i) a manual tube agglutination method, which is typically performed by a technologist at a lab bench; (ii) automated hemagglutination testing on an instrument which mimics what is done at the bench, but which does not have the ability to discern mixed field; or (iii) a gel which has some discriminating power. The forward typing test described herein is unique in that: (1) it is solid phase, and (2) it has been adjusted to not merely be a positive/negative result, but to be positive/mixed-field/negative result. Thus, the methods and tests described herein are distinct from both the existing current methods, and provide a new capability for users of automated instruments which do not employ gel technology for forward blood typing or minor antigen typing.

### Example 4

### Mixed-Field DAT Detection Capability

### Background

Direct Agglutination Test (DAT) is a common blood typing test. This test is used to identify if a sample of red blood cells (RBCs) have IgG-class antibodies present on the surface of the cells. This may occur for a variety of reasons, including: (1) a HTR (hemolytic transfusion reaction); (2) a DHTR (delayed hemolytic transfusion reaction); (3) coating of a newborn's RBCs with IgG-class antibodies from a mother, such as anti-K, anti-E, or other IgG-class antibodies to major or minor antigens, which are present on the newborn's RBCs but which the mother has formed IgG-class antibodies to; (4) autoantibodies which attach to one's own cells including cases of AIHA (auto-immune hemolytic anemia); (5) non-HTR cases where shortened cell life occurs due to antibodies from the patient coating transfused cells; (6) drug-induced non-specific binding of antibodies to a person's RBCs; (7) other non-specific binding of antibodies to a person's RBCs, or (8) other causes/situations.

A DAT test is routinely performed manually in a tube. It is typically carried out in a test tube by washing the RBCs, incubating the washed RBCs with AHG (anti-human globulin), and then testing for hemagglutination, such as through centrifugation and visual observation through gentle disruption of the cell pellet. An expert blood banker can look for and identify mixed field results in DAT tests when doing manual tube tests.

It is generally believed that gel technology can pick up mixed field in DAT testing, and that automated hemagglutination systems cannot. Because DAT testing is important to identify various important medical conditions of the patient and possible negative situations, proper DAT testing, including reporting situations where mixed field occurs is important.

Described herein is a solid phase testing method wherein a DAT is performed and the test result is determined as positive, negative, or mixed field.

### Experimental Preparation and Test:

The surface of a conical, round-bottom, flat-bottom, substantially planar, or similar well is coated with AHG (possibly a blend, or possibly of a certain specificity—for example, anti-C3D, anti-IgG, or a blend of both) antibodies.

Whole blood to be tested is washed and then diluted (ballpark of 0.2% dilution, see the Examples above).

A volume of sample is introduced such that the cells, if layered onto the surface, would form less than a monolayer (see the Examples above). It may be preferable to use a lower total number of cells than those exemplified above, for example, down to 1% or less of the number of cells specified in the Examples above.

The cells are centrifuged to the surface, as described in the above Examples. The exact centrifugation conditions can vary depending on the shape of the well chosen, the characteristics of the sample and the particulars of the substrate (degree of functional groups). For example, the cells can be centrifuged in one or more steps of relative short duration, e.g., 1 to 3 (e.g., about 1.5) minutes each, each one at 300-800 g (e.g., about 500 g). Alternatively, the cells can be centrifuged for longer periods, e.g., 5 to 10 (e.g., 8) minutes, at 25 to 100 g (e.g., about 50 g). Any combination of centrifugation conditions can be performed.

Cells which have antibodies of the given class (or otherwise which the AHG is specific to) on their surface will bind to the surface. Cells without antibodies of the given class (or otherwise which the AHG is specific to) will not detectably bind to the surface. The low overall coverage of the surface will enable unbound cells to move to the lowest point, as the bound cells do not form a tight carpet and will generally not strongly impede the migration of unbound cells down the surface.

The wells are imaged to determine the test result. Alternately, a non-imaging method may be employed to identify the presence, absence, or quantity of cells at different locations on the well surface. The results are interpreted as follows:

Pellet and very few or no cells on angled surfaces, indicating that the red blood cells do not have antibodies of the given class (or otherwise which the AHG is specific to) on their surface;

Many cells on angled surfaces and no pellet, indicating that the red blood cells have antibodies of the given class (or otherwise which the AHG is specific to) on their surface;

Both pellet and many cells on angled surfaces, indicating a mixed field, e.g., having two populations of red blood cells, a population with antibodies on the surface of the given specificity and a population without antibodies on the surface of the given specificity; or

If no cells are detected anywhere, it may indicate an error in, e.g., pipetting sample or centrifuging, or other error.

### Example 5

### Solid Substrate Coombs Crossmatch Testing

### Background

To ensure compatibility between a transfusable unit of red blood cells and a patient, the blood must be major blood type compatible and also the red cells must be compatible with regard to the absence of red cell antigens for which the patient presents clinically significant antibodies. This compatibility is generally ensured through a redundant testing process. In the first layer of compatibility testing, the patient is tested for clinically significant antibodies (i.e. antibody screening test) and if clinically significant alloantibodies exist, they are identified (i.e. antibody identification tests) and the transfusable unit is confirmed to be antigen-negative for the given clinically significant alloantibodies. A second layer of testing provides a layer of redundancy to mitigate the odds of error and also enables the detection of less likely events, such as alloimmunization against red cell antigens which are less frequently observed at levels of significance. The second layer of testing, commonly referred to as “Coombs Crossmatch” or “TAT Crossmatch” involves: (1) the incubation of transfusable red cells with patient plasma at 37 C to enable patient antibodies to bind to red cell antigens to which they are specific to, and (2) the detection of the presence of IgG-class antibodies on the surface of the cells, which is commonly performed by performing a hemagglutination test using anti-IgG to enable the agglutination of red blood cells with IgG-class antibodies on their surface.

Described herein is a solid phase testing method wherein a Coombs Crossmatch is performed.

### Experimental Preparation and Test:

A small quantity of transfusable red blood cells is first washed to remove antibodies and hemoglobin. These cells are resuspended, such as to create a 3% suspension. The resuspended washed donor cells are mixed into test plasma at a 1:2 or 1:4 ratio. PEG is added in a quantity equal to the amount of test plasma used, and the solution is mixed. The mixture is incubated at 37 C for 15 to 30 minutes. These cells are then washed into saline and resuspended at least 3 times. The final resuspension should produce a 0.2% solution of red cells.

The final resuspension of red cells are then tested according to the procedure above for DAT testing. For example the surface coating may employ Alba polyclonal monospecific anti-IgG (product code Z356). A positive test result indicates that antibodies are present of the class specificity of the reagent used on the surface. For example, a positive result may indicate the presence of IgG class antibodies in the test plasma with specificities to antigens on the transfusable red blood cells. A negative test result, seen as a pellet, indicates the absence or very low number of such IgG class antibodies in the test plasma with specificity to antigens on the transfusable red blood cells.

TABLE 1
Blood group antigens within systems
. . . = obsolete; provisional numbers are in italic

| | | Antigen Number | | | | | | | | | | | |
|---|---|---|---|---|---|---|---|---|---|---|---|---|---|
| System | | 001 | 002 | 003 | 004 | 005 | 006 | 007 | 008 | 009 | 010 | 011 | 012 |
| 001 | ABO | A | B | A, B | A1 | . . . | | | | | | | |
| 002 | MNS | M | N | S | s | U | He | Mi^{a} | M^{c} | Vw | Mur | M^{g} | Vr |
| 003 | P | P1 | . . . | . . . | | | | | | | | | |
| 004 | RH | D | C | E | c | e | F | Ce | C^{w} | C^{x} | V | E^{w} | G |
| 005 | LU | Lu^{a} | Lu^{b} | Lu3 | Lu4 | Lu5 | Lu6 | Lu7 | Lu8 | Lu9 | . . . | Lu11 | Lu12 |
| 006 | KEL | K | k | Kp^{a} | Kp^{b} | Ku | Js^{a} | Js^{b} | . . . | . . . | Ul^{a} | K11 | K12 |
| 007 | LE | Le^{a} | Le^{b} | Le^{ab} | Le^{bH} | ALe^{b} | BLe^{b} | | | | | | |
| 008 | FY | Fy^{a} | Fy^{b} | Fy3 | Fy4 | Fy5 | Fy6 | | | | | | |
| 009 | JK | Jk^{a} | Jk^{b} | Jk3 | | | | | | | | | |
| 010 | DI | Di^{a} | Di^{b} | Wr^{a} | Wr^{b} | Wd^{a} | Rb^{a} | WARR | ELO | Wu | Bp^{a} | Mo^{a} | Hg^{a} |
| 011 | YT | Yt^{a} | Yt^{b} | | | | | | | | | | |
| 012 | XG | Xg^{a} | CD99 | | | | | | | | | | |
| 013 | SC | Sc1 | Sc2 | Sc3 | Rd | STAR | SCER | SCAN | | | | | |
| 014 | DO | Do^{a} | Do^{b} | Gy^{a} | Hy | Jo^{a} | DOYA | | | | | | |
| 015 | CO | Co^{a} | Co^{b} | Co3 | | | | | | | | | |
| 016 | LW | . . . | . . . | . . . | . . . | LW^{a} | LW^{ab} | LW^{b} | | | | | |
| 017 | CH/RG | Ch1 | Ch2 | Ch3 | Ch4 | Ch5 | Ch6 | WH | | | | Rg1 | Rg2 |
| 018 | H | H | | | | | | | | | | | |
| 019 | XK | Kx | | | | | | | | | | | |
| 020 | GE | . . . | Ge2 | Ge3 | Ge4 | Wb | Ls^{a} | An^{a} | Dh^{a} | GEIS | | | |
| 021 | CROM | Cr^{a} | Tc^{a} | Tc^{b} | Tc^{c} | Dr^{a} | Es^{a} | IFC | WES^{a} | WES^{b} | UMC | GUTI | SERF |
| 022 | KN | Kn^{a} | Kn^{b} | McC^{a} | Sl1 | Yk^{a} | McC^{b} | Sl2 | Sl3 | KCAM | | | |
| 023 | IN | In^{a} | In^{b} | INFI | INJA | | | | | | | | |
| 024 | OK | Ok^{a} | | | | | | | | | | | |
| 025 | RAPH | MER2 | | | | | | | | | | | |
| 026 | JMH | JMH | JMHK | JMHL | JMHG | JMHM | | | | | | | |
| 027 | I | I | | | | | | | | | | | |
| 028 | GLOB | P | | | | | | | | | | | |
| 029 | GIL | GIL | | | | | | | | | | | |
| 030 | RHAG | Duclos | Ol^{a} | Ducols- | | | | | | | | | |
| | | | | like | | | | | | | | | |

| | | Antigen number | | | | | | | | | | | |
|---|---|---|---|---|---|---|---|---|---|---|---|---|---|
| System | | 013 | 014 | 015 | 016 | 017 | 018 | 019 | 020 | 021 | 022 | 023 | 024 |
| 002 | MNS | M^{e} | Mt^{a} | St^{a} | Ri^{a} | Cl^{a} | Ny^{a} | Hut | Hil | M^{v} | Far | s^{D} | Mit |
| 004 | RH | . . . | . . . | . . . | . . . | Hrₒ | Hr | hr^{S} | VS | C^{G} | CE | D^{w} | . . . |
| 005 | LU | Lu13 | Lu14 | . . . | Lu16 | Lu17 | Au^{a} | Au^{b} | Lu20 | Lu21 | | | |
| 006 | KEL | K13 | K14 | . . . | K16 | K17 | K18 | K19 | Km | Kp^{c} | K22 | K23 | K24 |
| 010 | DI | Vg^{a} | Sw^{a} | BOW | NFLD | Jn^{a} | KREP | Tr^{a} | Fr^{a} | SW1 | | | |
| 021 | CROM | ZENA | CROV | CRAM | | | | | | | | | |

| | | Antigen number | | | | | | | | | | |
|---|---|---|---|---|---|---|---|---|---|---|---|---|
| System | | 025 | 026 | 027 | 028 | 029 | 030 | 031 | 032 | 033 | 034 | 035 |
| 002 | MNS | Dantu | Hop | Nob | En^{a} | En^{a}KT | ‘N’ | Or | DANE | TSEN | MINY | MUT |
| 004 | RH | . . . | c-like | cE | hr^{H} | Rh29 | Go^{a} | hr^{B} | Rh32 | Rh33 | Hr^{B} | Rh35 |
| 006 | KEL | VLAN | TOU | RAZ | VONG | KALT | KTIM | KYO | KUCI | KANT | KASH | |

| | | Antigen number | | | | | | | | | | |
|---|---|---|---|---|---|---|---|---|---|---|---|---|
| System | | 036 | 037 | 038 | 039 | 040 | 041 | 042 | 043 | 044 | 045 | 046 |
| 002 | MNS | SAT | ERIK | Os^{a} | ENEP | ENEH | HAG | ENAV | MARS | ENDA | ENEV | MNTD |
| 004 | RH | Be^{a} | Evans | . . . | Rh39 | Tar | Rh41 | Rh42 | Crawford | Nou | Riv | Sec |

| | | Antigen number | | | | | | | | | | |
|---|---|---|---|---|---|---|---|---|---|---|---|---|
| System | | 047 | 048 | 049 | 050 | 051 | 052 | 053 | 054 | 055 | 056 | 057 |
| 004 | RH | Dav | JAL | STEM | FPTT | MAR | BARC | JAHK | DAK | LOCR | CENR | CEST |

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of preparing and/or evaluating a sample for a mixed population of red blood cells (RBCs), a sample comprising a first population of RBCs and a second population of RBCs, wherein cells of said first population comprise a surface feature, e.g., a surface antigen, and cells of said second population are substantially devoid of said surface feature,
said method comprising
(a) providing a capture agent disposed on a substrate,
wherein the capture agent binds the surface feature, e.g., an antigen on cells of the first population but not cells of the second population, and
wherein the substrate is configured such that cells that do not bind to the capture-agent migrate to a negative readout region (NRR);
(b) contacting the sample with the capture agent under conditions sufficient for binding, e.g., the formation of an immune complex, between the capture agent and a cell of the first population of cells, which binding occur, e.g., in a positive readout region (PRR);
(c) providing conditions that allow cells of the of the second population that do not bind to the capture agent to migrate into a NRR;
(d) classifying the PRR as positive or non-positive for presence of cells;
(e) classifying the NRR as positive or non-positive for the presence of cells;
wherein,
(i) PRR+ and NRR− is indicative of the sample lacking the second population;
(ii) PRR− and NRR+ is indicative of a sample lacking the first population;
(iii) PRR+ and NRR+ is indicative of a sample having both the first population and the second population,
thereby preparing and/or evaluating a sample for a mixed population of RBCs.

2. A method of preparing and/or evaluating a sample for a mixed population of RBCs, e.g., a sample comprising a first population of RBCs and a second population of RBCs, wherein cells of said first population comprise a surface feature, e.g.; a surface antigen, and cells of said second population are substantially devoid of said surface feature,
said method comprising
(a) providing a capture agent disposed on a substrate,
wherein the capture agent binds the surface feature, e.g., an antigen on cells of the first population but not cells of the second population, and
wherein the substrate is configured such that cells that do not bind to the capture agent migrate to a negative readout region (NRR);
(b) contacting the sample with the capture agent under conditions sufficient for binding, e.g., the formation of an immune complex, between the capture agent and a cell of the first population of cells, which binding occur, e.g., in a positive readout region (PRR);
(c) providing conditions that allow cells of the of the second population that do not bind to the capture agent to migrate into a NRR;
(d) performing one or both of
(i) classifying the PRR as:
single or high positive (which is indicative of a sample lacking the second population);
mixed or low positive (which is indicative of a sample having both the first and second populations); or
negative (which is indicative of a sample lacking the first population); or
(ii) classifying the NRR as:
single or high positive (which is indicative of a sample lacking first population);
mixed or low positive (which is indicative of a sample having both the first and second populations); or
negative (which is indicative of a sample lacking the second population)
thereby preparing and/or evaluating a sample, e.g., for a mixed population of RBCS.

3. The method of claim 1, wherein the method includes evaluating the sample for the presence of a population having a second surface feature and a population devoid of, or substantially devoid of, the second surface feature.

4. The method of claim 1, wherein the surface feature present on the cells of the first population and not present on, or substantially devoid from, the cells of the second population is a surface antigen from Table 1.

5. The method of claim 1, wherein the surface feature present on the cells of the first population and not present on, or substantially devoid from, the cells of the second population is a RBC blood group antigen, e.g., A, B or D.

6. The method of claim 5, wherein the capture agent comprises an antibody specific for the RBC blood antigen, e.g., for an A, B or D antigen.

7. The method of claim 1, wherein the surface feature present on the cells of the first population and not present on, or substantially devoid from, cells of the second population is a minor RBC blood group antigen.

8. The method of claim 7, wherein the antigen is; a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl.

9. The method of claim 1, wherein the sample is from a subject that has had a blood transfusion, e.g., a recent blood transfusion, e.g., a transfusion within 1, 5, 10, 20, 30 60, or 90 days of taking of the sample.

10. The method of claim 1, wherein the sample is from a subject that has had a bone marrow, or other tissue, transplant.

11. The method of claim 1, wherein the sample is from a subject that is a hematopoietic chimera, e.g., a subject having naturally occurring chimerism (e.g., arising from the fusion of more than one embryo into a single embryo or the exchange of cells between embryos) or having chimerism arising from a transplant.

12. The method of claim 1, wherein the sample is tested for a first population of cells having IgG antibodies bound to the surface of the cells and a second population of cells that are devoid, or substantially devoid of IgG antibodies bound to the surface.

13. The method of claim 12, wherein the capture agent comprises an antibody, e.g., an anti-human globulin (AHG).

14. The method of claim 12, wherein the capture agent comprises one or both of anti-C3D and anti-IgG antibodies.

15. The method of claim 12, wherein the sample: (1) is from a subject that has had a HTR (hemolytic transfusion reaction), (2) is from a subject that has had a DHTR (delayed hemolytic transfusion reaction), (3) is from a newborn having its RBCs coated with IgG-class antibodies from the mother, such as anti-K, anti-E, or other IgG-class antibodies to major or minor antigens which are present on the newborn's RBCs but which the mother has formed IgG-class antibodies to, (4) is from a subject that has autoantibodies which attach to it's own cells, (5) is from a subject other than one with HTR, where shortened cell life occurs due to antibodies from the subject coating transfused cells, (6) is from a subject that has drug-induced non-specific binding of antibodies to a its's RBCs, (7) is from a subject that has other non-specific binding of antibodies to a it's RBCs, or (8) is from a subject that has other causes of anti-RBC antibodies.

16. The method of claim 12, wherein the surface feature present on the cells of the first population and not present on, or substantially devoid from, the cells of the second population is an antibody that binds A, B or D antigen.

17. The method of claim 12, wherein the surface feature present on the cells of the first population and not present on, or substantially devoid from, cells of the second population is an antibody to a minor RBC blood group antigen.

18. The method of claim 17, wherein the antigen is: a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment, the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. Thus, the method can detect the presence or absence of populations having a different minor RBC blood group antigen.

19. The method of claim 1, wherein the amount of sample cells contacted with the substrate is such that, if layered onto the substrate, e.g., the portion of the substrate coated with capture agent, it would form less than a monolayer of sample cells.

20. The method of claim 1, wherein positive and negative readout regions are imaged to provide a result.

21. The method of claim 1, wherein the substrate is configured such that the applied acceleration allows cells of the first population to be distinguished from cells of the second population.

22. The method of claim 1, wherein the substrate is a substantially planar substrate.

23. A method of evaluating a sample comprising red blood cells, e.g., evaluating a sample for the presence of antibody, e.g., IgG antibody, bound to the surface of said red blood cells, said method comprising:
(a) providing a capture agent disposed on a substrate,
wherein said capture agent binds a human antibody, e.g., IgG antibody, e.g., wherein said capture agent is a AHG antibody, and wherein the substrate is configured such that cells lacking antibodies reactive with the capture agent bound to their surfaces, and thus that do not bind to the capture agent, migrate to a negative readout region (NRR);
(b) contacting said sample with the capture agent under conditions sufficient for binding, e.g., the formation of an immune complex, between the capture agent and an antibody reactive with the capture agent bound to a red blood cell, e.g., to a red blood cell surface antigen, in said sample, which binding occurs, e.g., in a positive readout region (PRR), and, e.g., sufficient to allow cells lacking antibodies reactive with the capture agent bound to their surfaces, and thus that do not bind to the capture agent to migrate to said NRR;
thereby evaluating a sample for red blood cells for the presence of antibody, e.g., IgG antibody, bound to the surface of said red blood cells.

24. The method of claim 23, wherein said sample comprises red blood cells which may have antibodies bound thereto and wherein said red blood cells and said antibodies are from the same person

25. The method of claim 24, wherein said evaluation comprises a direct agglutination test.

26. The method of claim 23, wherein said sample comprises plasma from a person and red blood cells that are not from said person, e.g., red cells from another person or reagent or reference red blood cells.

27. The method of claim 26, wherein said antibodies from said plasma are bound to said red blood cells.

28. The method of claim 26, wherein said evaluation comprises an indirect agglutination test.

29. The method of claim 23, further comprising:
classifying the PRR as positive or non-positive for presence of cells (In an embodiment, this can be done by determining if the number of cells in the PRR meets a predetermined reference value(s)).

30. The method of claim 23, further comprising:
classifying the NRR as positive or non-positive for presence of cells (In an embodiment, this can be done by determining if the number of cells in the NRR meets a predetermined reference value(s)).

31. The method of claim 23, wherein one or both of a PRR positive score and/or a negative NRR score is obtained and said sample is classified as having red blood cells with an antibody bound thereto.

32. The method of claim 23, wherein one or both of a PRR negative score and/or a NRR positive score is obtained and said sample is classified as not having red blood cells with an antibody bound thereto.

33. The method of claim 23, wherein said sample comprises red cells from a subject and antibodies bound thereto from said subject.

34. The method of claim 28, wherein responsive to the evaluation, a patient is classified as having immune hemolytic anemia or not having immune hemolytic anemia.

35. The method of claim 23, wherein one or both of a PRR positive score and/or a negative NRR score is obtained and said subject is classified as having immune hemolytic anemia.

36. The method of claim 28, wherein one or both of a PRR negative score and/or a NRR positive score is obtained and said subject is classified as not having immune hemolytic anemia.

37. The method of claim 23, wherein said sample comprises a component from a first subject, e.g., a candidate recipient, and a second subject, e.g., a candidate donor.

38. The method of claim 37, wherein said sample comprises plasma from said first subject and red blood cells from said second subject.

39. The method of claim 37, wherein responsive to the evaluation, a blood product from said second subject is determined to be compatible for transfusion to said first subject.

40. The method of claim 37, wherein one or both of a PRR positive score and/or a negative NRR score is obtained and said patient is not approved for receiving a product comprising red blood cells from a donor.

41. The method of claim 37, wherein one or both of a PRR negative score and/or a NRR positive score is obtained and said patient is approved for receiving a product comprising red blood cells from a donor.

42. The method of claim 37, wherein responsive to the evaluation, a donor product comprising red blood cells is approved or not approved administration to said patient.

43. The method of claim 42, wherein one or both of a PRR positive score and/or a negative NRR score is obtained and said donor product is not approved for administration to said patient.

44. The method of claim 42, wherein one or both of a PRR negative score and/or a NRR positive score is obtained and said donor product is approved for administration to said patient.

45. The method of claim 23, wherein said antibody bound to a red blood cell, e.g., to a red blood cell surface antigen, is specific for a RBC blood antigen, e.g., for an A, B or D antigen.

46. The method of claim 23, wherein said antibody bound to a red blood cell, e.g., to a red blood cell surface antigen, is specific for a minor RBC blood group antigen.

47. The method of claim 23, wherein said antibody bound to a red blood cell, e.g., to a red blood cell surface antigen, is specific for a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl. In an embodiment the capture agent, e.g., an antibody, is specific for the minor RBC blood group antigen, a Rhesus antigen, e.g., D, C, c, E, or e; a MNS antigen, e.g., M, N, S, or s; a Kidd antigen, e.g., Jk^{a} or Jk^{b}; a Duffy antigen, e.g., Fy^{a} or Fy^{b}; a Kell antigen, e.g., K or k; a Lewis antigen, e.g., Le^{a} or Le^{b}; or P antigen, e.g., Pl.

48. The method of claim 23, wherein said capture agent comprises an antibody, e.g., an anti-human globulin (AHG).

49. The method of claim 23, wherein said capture agent comprises one or both of anti-C3D and anti-IgG antibodies.

50. The method of claim 23, wherein the sample; (1) is from a subject that has had a HTR (hemolytic transfusion reaction), (2) is from a subject that has had a DHTR (delayed hemolytic transfusion reaction), (3) is from a newborn having its RBCs coated with IgG-class antibodies from the mother, such as anti-K, anti-E, or other IgG-class antibodies to major or minor antigens which are present on the newborn's RBCs but which the mother has formed IgG-class antibodies to, (4) is from a subject that has autoantibodies which attach to it's own cells, (5) is from a subject other than one with HTR, where shortened cell life occurs due to antibodies from the subject coating transfused cells, (6) is from a subject that has drug-induced non-specific binding of antibodies to a it's RBCs, (7) is from a subject that has other non-specific binding of antibodies to a it's RBCs, or (8) is from a subject that has other causes of anti-RBC antibodies.

51. The method of claim 23, wherein positive and negative readout regions are imaged to provide a result.

52. The method of claim 23, wherein the substrate is a substantially planar substrate.

53. The method of claim 23, comprising;
(d) performing one or both of
(i) classifying the PRR as:
single or high positive (which is indicative of a sample in which all or substantially all cells are bound by an antibody);
mixed or low positive (which is indicative of a sample having both a population of cells bound by an antibody and a population of cells not bound by an antibody); or
negative (which is indicative of a sample lacking cells bound by an antibody); or,
(ii) classifying the NRR as:
single or high positive (which is indicative of a sample lacking cells bound by an antibody);
mixed or low positive (which is indicative of a sample having both a population of cells bound by an antibody and a population of cells not bound by an antibody); or
negative (which is indicative of a sample having a population of cells bound by an antibody).

54. The method of claim 23 comprising:
(i) classifying a sample with a positive PRR and a negative NRR as positive (which is indicative of a sample in which all or substantially all cells are bound by an antibody);
(ii) classifying a sample with a negative PRR and a positive NRR as negative (which is indicative of a sample wherein all or substantially all cells are not bound by antibodies for which the capture agent is specific to);
(iii) classifying a sample with a positive PRR and a positive NRR as mixed field (which is indicative of a sample having both a population of cells bound by an antibody for which the capture agent is specific and a population of cells not bound by an antibody for which the capture agent is specific).

55. The method of claim 28 wherein the sample is reported has giving a mixed field result, indicative of having both a population of cells bound by an antibody for which the capture agent is specific and a population of cells not bound by an antibody for which the capture agent is specific.
